(19) United States
(12) Patent Application Publication
(10) Pub. No.: US 2021/0395760 A1
(43) Pub. Date: Dec. 23, 2021

(54) COMPOSITIONS AND METHODS FOR OCHROBACTRUM-MEDIATED GENE EDITING

(71) Applicant: PIONEER HI-BRED INTERNATIONAL, INC., JOHNSTON, IA (US)

(72) Inventors: JYOTHI BOLAR, GRIMES, IA (US); SHUJUN CHANG, JOHNSTON, IA (US); HYEON-JE CHO, ANKENY, IA (US); ZHAN-BIN LIU, CLIVE, IA (US)

(73) Assignee: PIONEER HI-BRED INTERNATIONAL, INC., JOHNSTON, IA (US)

(21) Appl. No.: 17/288,902

(22) PCT Filed: Oct. 30, 2019
(86) PCT No.: PCT/US2019/058750

§ 371 Date: Apr. 26, 2021

Related U.S. Application Data
(60) Provisional application No. 62/753,577, filed on Oct. 31, 2018.

(51) Int. Cl. C12N 15/82; C12N 9/22; C12N 15/11

(52) U.S. Cl. CPC C12N 15/8213; C12N 9/22; C12N 2310/20; C12N 15/8205; C12N 15/8241; C12N 15/111

(57) ABSTRACT

Methods and compositions for increasing, improving or enhancing gene editing efficiency are provided. Configurations of Ochrobactrum and Agrobacterium based vector components such as CRISPR Cas endonucleases and guide RNAs are provided that improve efficiency of targeted genome modification.

### CROSS-REFERENCE TO RELATED APPLICATIONS

This application claims the benefit of PCT Application Serial Number PCT/US2019/058750, filed Oct. 30, 2019, which claims the benefit of U.S. Provisional Patent Application Ser. No. 62/753,577 filed 31 Oct. 2018, which is herein incorporated by reference in its entirety.

### FIELD OF THE DISCLOSURE

The present disclosure relates to the field of plant molecular biology, in particular to compositions and methods for modifying the genome of a cell

### REFERENCE TO SEQUENCE LISTING SUBMITTED ELECTRONICALLY

The official copy of the sequence listing is submitted electronically via EFS-Web as an ASCII formatted sequence listing with a file named “7789-US-PCT_ST25” created on Apr. 23, 2021 and having a size of 603 kilobytes and is filed concurrently with the specification. The sequence listing contained in this ASCII formatted document is part of the specification and is herein incorporated by reference in its entirety.

### BACKGROUND OF THE DISCLOSURE

Recombinant DNA technology has made it possible to insert DNA sequences at targeted genomic locations and/or modify specific endogenous chromosomal sequences. Site-specific integration techniques, which employ site-specific recombination systems, as well as other types of recombination technologies, have been used to generate targeted insertions of genes of interest in a variety of organisms. Genome-editing techniques such as designer zinc finger nucleases (ZFNs), transcription activator-like effector nucleases (TALENs), or homing meganucleases, are available for producing targeted genome perturbations, but these systems tend to have low specificity and employ designed nucleases that need to be redesigned for each target site, which renders them costly and time-consuming to prepare.

Newer technologies utilizing archaeal or bacterial adaptive immunity systems have been identified, called CRISPR (Clustered Regularly Interspaced Short Palindromic Repeats), which comprise different domains of effector proteins that encompass a variety of activities (DNA recognition, binding, and optionally cleavage).

Despite the identification and characterization of some of these systems, there remains a need for identifying novel effectors and systems, as well as demonstrating activity in eukaryotes, particularly animals and plants, to effect improved editing of endogenous and previously-introduced heterologous polynucleotides and for methods and compositions for improving the frequency and efficiency of homology-directed repair of double-strand-break sites.

### SUMMARY OF THE DISCLOSURE

In an aspect, a method for increasing the efficiency of editing a target site in a plant, the method comprising (a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary to the target site and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage at the target site; (b) identifying at least one plant cell of (a) that has a modification at the target site, wherein the modification includes at least one deletion or substitution of one or more nucleotides at the target site; and (c) regenerating a plant from the at least one plant cell of (b) having the modification at the target site having increased editing efficiency when compared to a control plant having a modification at the target site provided by a control plant editing T-DNA, wherein the control plant editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary to the target site and wherein the guide RNA and the Cas endonuclease are capable of forming a complex that enables the Cas endonuclease to introduce cleavage at the target site is provided.

In an aspect, a method of increasing the efficiency of altering the fatty acid profile in the seed of a plant, the method comprising (a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage in the FAD2 genomic sequence in the plant genome, the FAD3 genomic sequence in the plant genome, or a combination thereof; (b) obtaining a plant from the plant cell of (a); (c) evaluating the plant of (b) for the presence of the at least one nucleotide modification; (d) selecting a progeny plant of (c) having an altered fatty acid profile; and (e) obtaining seed from the progeny plant of (d) having increased editing efficiency when compared to a control seed of a plant having a modification of the FAD2 genomic sequence, the FAD3 genomic sequence, or the combination thereof provided by a control seed editing T-DNA, wherein the control seed editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof wherein the guide RNA and the Cas endonuclease are capable of forming a complex that enables the Cas endonuclease to introduce cleavage in the FAD2 genomic sequence in the plant genome, the FAD3 genomic sequence in the plant genome, or a combination thereof is provided.

In an aspect, a method of increasing the efficiency of altering the fatty acid profile in the seed of a plant, the method comprising (a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage within the FAD2 genomic sequence in the plant genome, the FAD3 genomic sequence in the plant genome, or a combination thereof; (b) obtaining a plant from the plant cell of (a); (c) evaluating the plant of (b) for the presence of the at least one nucleotide modification; (d) screening a progeny plant of (c) having an altered fatty acid profile that is void of the guide RNA and the Cas endonuclease; and (e) obtaining seed from the progeny plant of (d) having increased editing efficiency when compared to a control seed of a plant having a modification of the FAD2 genomic sequence, the FAD3 genomic sequence, or the combination thereof provided by a control seed editing T-DNA, wherein the control seed editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and wherein the guide RNA and the Cas endonuclease are capable of forming a complex that enables the Cas endonuclease to introduce cleavage within the FAD2 genomic sequence in the plant genome, the FAD3 genomic sequence in the plant genome, or a combination thereof is provided.

In an aspect, a method for increasing the efficiency of introducing a nucleotide of interest into a target site in the genome of a plant, the method comprising (a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to cleavage at the target site; (b) contacting the plant cell of (a) with a donor DNA comprising the polynucleotide of interest; (c) identifying at least one plant cell from (b) comprising in its genome the polynucleotide of interest integrated at the target site; and (d) regenerating a plant from the at least one plant cell having in its genome the polynucleotide of interest integrated at the target site having increased efficiency of introduction of the polynucleotide of interest into the target site in the genome of the plant cell when compared to a control plant having an introduction of the polynucleotide of interest into the target site in the genome of the plant cell provided by a control plant editing T-DNA, wherein the control plant editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA and the Cas endonuclease are capable of forming a complex that enables the Cas endonuclease to cleavage at the target site is provided. In an aspect, the method further comprising a trait of interest or a nucleotide modification template cassette, wherein the trait of interest or nucleotide modification template cassette comprises at least one nucleotide modification of the trait of interest or nucleotide and the trait of interest or nucleotide modification template cassette is capable of making at the least one nucleotide modification at the target site of the trait of interest or the nucleotide. In an aspect, the method further comprising a modification template cassette, wherein the modification template cassette comprises at least one nucleotide modification of a FAD2 genomic sequence in the plant genome, a FAD3 genomic sequence in the plant genome, or a combination thereof and the modification template cassette enables the at least one nucleotide modification of the FAD2 genomic sequence in the plant genome, the FAD3 genomic sequence in the plant genome, or the combination thereof. In an aspect, the target site is selected from the group consisting of a promoter sequence, a terminator sequence, a regulatory element sequence, a coding sequence, a splice site, a polyubiquitination site, an intron site, an intron enhancing motif, a gene of interest, and a trait of interest. In an aspect, the target site is selected from the group consisting of a polynucleotide encoding selectable marker resistance, disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein composition, altered oil composition, increased biomass, increased shoot length, increased root length, improved root architecture, modulation of a metabolite, modulation of the proteome, increased seed weight, altered seed carbohydrate composition, altered seed oil composition, altered fatty acid profile, altered seed protein composition, altered seed nutrient composition, improved fertility, improved fecundity, improved environmental tolerance, improved vigor, improved disease resistance, improved disease tolerance, improved tolerance to a heterologous molecule, improved fitness, improved physical characteristic, greater mass, increased production of a biochemical molecule, decreased production of a biochemical molecule, upregulation of a gene, downregulation of a gene, upregulation of a biochemical pathway, downregulation of a biochemical pathway, stimulation of cell reproduction, and suppression of cell reproduction. In an aspect, the polynucleotide encodes an altered fatty acid profile. In an aspect, the plant is a monocot or a dicot. In as aspect, the monocot is selected from the group consisting of maize, rice, sorghum, rye, barley, wheat, millet, oats, sugarcane, turfgrass, and switchgrass. In an aspect, the dicot is selected from the group consisting of soybean, canola, alfalfa, sunflower, cotton, tobacco, peanut, potato, tobacco, Arabidopsis, and safflower. In an aspect, the editing T-DNA provided to the plant cell is provided via Agrobacterium-mediated transformation. In an aspect, the editing T-DNA provided to the plant cell is provided via Ochrobactrum-mediated transformation. In an aspect, the editing T-DNA provided to the plant cell is provided via Rhizobiaceae-mediated transformation. In an aspect, the guide RNA is operably linked to a plant U6 polymerase III promoter. In an aspect, the Cas endonuclease is a plant optimized Cas9 endonuclease. In an aspect, the Cas endonuclease gene is operably linked to a nuclear targeting signal upstream of the Cas coding region and a nuclear localization signal downstream of the Cas coding region. In an aspect, the target site is located in the gene sequence of a FAD2 gene, a FAD3 gene, or a combination thereof. In an aspect, a plant, plant cell, or seed produced by the methods provided herein. In an aspect, a plant comprising an edited trait of interest, wherein the plant originates from a plant cell comprising an edited trait of interest produced by the methods provided herein. In an aspect, wherein the editing T-DNA further comprises a selectable marker expression cassette, a color marker expression cassette, or a combination thereof. In an aspect, the Cas endonuclease is expressed by SEQ ID NO:1.

In an aspect, a recombinant DNA construct for increasing editing comprising an editing T-DNA for a trait or polynucleotide of interest, wherein the editing T-DNA comprises in operable linkage from a right border to a left border orientation, a guide RNA expression cassette, wherein the guide RNA expression cassette comprises a regulatory element operably linked to a polynucleotide encoding a guide RNA, wherein the guide RNA is complementary to the trait or polynucleotide of interest and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease cleavage in the trait or polynucleotide of interest is provided. In an aspect, the recombinant DNA construct further comprising a trait or polynucleotide of interest modification template cassette, wherein the trait or polynucleotide of interest modification template cassette comprises at least one nucleotide modification of the trait or polynucleotide of interest and the trait or polynucleotide of interest modification template cassette is capable of making at the least one nucleotide modification in the trait or polynucleotide of interest. In an aspect, the recombinant DNA construct further comprises a selectable marker expression cassette, a color marker expression cassette, or a combination thereof.

In an aspect, a plant comprising a modified nucleotide sequence, wherein the modified nucleotide sequence was produced by providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary the nucleotide sequence and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage in the nucleotide sequence is provided. In an aspect, the editing T-DNA further comprises a nucleotide modification template cassette, wherein the nucleotide modification template cassette comprises at least one nucleotide modification of the nucleotide sequence and the nucleotide modification template cassette is capable of making at the least one nucleotide modification in the nucleotide sequence.

In an aspect, a plant comprising a modified nucleotide sequence, wherein the modified nucleotide sequence was produced by providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage in the FAD2 genomic sequence in the plant genome, the FAD3 genomic sequence in the plant genome, or a combination thereof is provided. In an aspect, the editing T-DNA further comprises a polynucleotide modification template cassette, wherein the polynucleotide modification template cassette comprises at least one nucleotide modification of a FAD2 genomic sequence in the plant genome, a FAD3 genomic sequence in the plant genome, or a combination thereof, and the polynucleotide modification template cassette enables the at least one nucleotide modification of the FAD2 genomic sequence in the plant genome, the FAD3 genomic sequence in the plant genome, or the combination thereof.

In an aspect, the plant is a monocot or a dicot. In an aspect, the monocot is selected from the group consisting of maize, rice, sorghum, rye, barley, wheat, millet, oats, sugarcane, turfgrass, and switchgrass. In an aspect, the dicot is selected from the group consisting of soybean, canola, alfalfa, sunflower, cotton, tobacco, peanut, potato, tobacco, Arabidopsis, and safflower.

### DESCRIPTION OF THE FIGURES

The disclosure can be more fully understood from the following detailed description and the accompanying drawings and Sequence Listing that form a part of this application, which are incorporated herein by reference.

FIG. 1A shows a synthetic construct comprising editing components of binary vector RV019927 as described herein, for soybean transformation.

FIG. 1B shows a synthetic construct comprising editing components of binary vector RV019928 as described herein, for soybean transformation.

FIG. 1C shows a synthetic construct comprising editing components of binary vector RV019929 as described herein, for soybean transformation.

FIG. 1D shows a synthetic construct comprising editing components of binary vector RV019930 as described herein, for soybean transformation.

FIG. 2 shows gene editing efficiency with different binary vectors RV019927, RV019928, RV019929, and RV019930.

FIG. 3A shows the edits made to the FAD2-1A coding sequence as described herein, such as the RV019927 experiment.

FIG. 3B shows the edits made to the FAD2-1B coding sequence as described herein, such as the RV019927 experiment.

FIG. 4A shows the edits made to the FAD2-1A coding sequence as described herein, such as the RV019929 experiment.

FIG. 4B shows the edits made to the FAD2-1B coding sequence as described herein, such as the RV019929 experiment.

FIG. 5A shows the edits made to the FAD3a coding sequence as described herein, such as the RV019929 experiment.

FIG. 5B shows the edits made to the FAD3b coding sequence as described herein, such as the RV019929 experiment.

FIG. 6 depicts one example of a plurality of target site (TS) polynucleotide sequences flanking the donor DNA cassette. “POI” stands for “polynucleotide of interest”, which in some examples encoded a trait of interest, for example a trait of agronomic importance or interest.

FIG. 7 depicts schematic illustrations of different vectors and experimental strategies for the soybean HDR experiments. FIG. 7A depicts the experimental strategy for Vector 9. FIG. 7B depicts the experimental strategy for Vector 10. FIG. 7C depicts the experimental strategy for Vector 11. FIG. 7D depicts the experimental strategy for Vector 12. FIG. 7E depicts the experimental strategy for Vector 13. FIG. 7F depicts the experimental strategy for Vector 14. FIG. 7G depicts the experimental strategy for Vector 15. FIG. 7H depicts the experimental strategy for Vector 16. FIG. 7I depicts the experimental strategy for Vector 17.

FIG. 8 depicts soy transformation Vector 9 (SEQ ID NO:97), corresponding to the schematic of FIG. 7A.

FIG. 9 depicts soy transformation Vector 10 (SEQ ID NO:98), corresponding to the schematic of FIG. 7B.

FIG. 10 depicts soy transformation Vector 11 (SEQ ID NO:99), corresponding to the schematic of FIG. 7C.

FIG. 11 depicts soy transformation Vector 12 (SEQ ID NO:100), corresponding to the schematic of FIG. 7D.

FIG. 12 depicts soy transformation Vector 13 (SEQ ID NO:101), corresponding to the schematic of FIG. 7E.

FIG. 13 depicts soy transformation Vector 14 (SEQ ID NO:102), corresponding to the schematic of FIG. 7F.

FIG. 14 depicts soy transformation Vector 15 (SEQ ID NO:103), corresponding to the schematic of FIG. 7G.

FIG. 15 depicts soy transformation Vector 16 (SEQ ID NO:104), corresponding to the schematic of FIG. 7H.

FIG. 16 depicts soy transformation Vector 17 (SEQ ID NO:105), corresponding to the schematic of FIG. 7I.

FIG. 17 shows the vector design and experimental strategy and results for the soy HDR experiments for Vector 9. FIG. 17A depicts the vector design and experimental strategy. FIG. 17B shows the individual read results for the samples. FIG. 17C shows the normalized concentration of HDR copy reads.

FIG. 18 shows the vector design and experimental strategy and results for the soy HDR experiments for Vector 10. FIG. 18A depicts the vector design and experimental strategy. FIG. 18B shows the individual read results for the samples. FIG. 18C shows the normalized concentration of HDR copy reads.

FIG. 19 shows the vector design and experimental strategy and results for the soy SDN2 experiments for Vector 13. FIG. 19A depicts the vector design and experimental strategy. FIG. 19B shows the wild type soy target sequence (SEQ ID NO:106) and the donor DNA sequence for gRNA2 (SEQ ID NO:107). FIGS. 19C and 19D show sequencing verification of edits.

FIG. 20 shows the vector schematic and experimental strategy for the soy HDR experiments for Vector 12.

FIG. 21 shows the results for three reps of two different germplasm lines for Vector 14.

FIG. 22 shows the results for three reps of two different germplasm lines for Vector 15.

### DETAILED DESCRIPTION

The sequence descriptions (Table 1) summarize the Sequence Listing attached hereto, which is hereby incorporated by reference. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

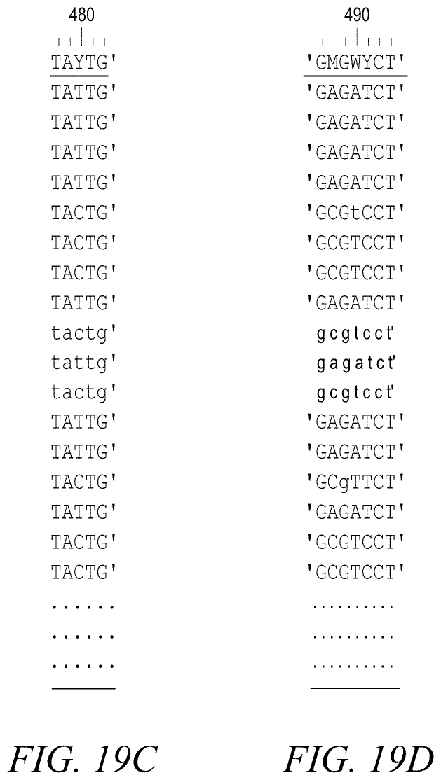

TABLE 1
Sequence Listing Description

| SEQ ID NO: | Description |
|---|---|
| SEQ ID NO: 1 | Nucleotide sequence of soybean codon optimized Cas9 |
| SEQ ID NO: 2 | Amino acid sequence of the AT-NLS nuclear localization signal |
| SEQ ID NO: 3 | Amino acid sequence of the VirD2 nuclear localization signal |
| SEQ ID NO: 4 | Nucleotide sequence of soybean constitutive promoter GM-EF1A2 |
| SEQ ID NO: 5 | Nucleotide sequence of soybean GM-U6-13.1 promoter |
| SEQ ID NO: 6 | Nucleotide sequence of soybean GM-U6-9.1 promoter |
| SEQ ID NO: 7 | Nucleotide sequence of GM-FAD2-1 CR1 |
| SEQ ID NO: 8 | Nucleotide sequence of GM-FAD3-CR2 |
| SEQ ID NO: 9 | Nucleotide sequence of RV019927 construct |
| SEQ ID NO: 10 | Nucleotide sequence of RV019929 construct |
| SEQ ID NO: 11 | Nucleotide sequence of FAD2-F1 primer |
| SEQ ID NO: 12 | Nucleotide sequence of FAD2-R1 primer |
| SEQ ID NO: 13 | Nucleotide sequence of FAD2-T1 probe |
| SEQ ID NO: 14 | Nucleotide sequence of FAD2-F2 primer |
| SEQ ID NO: 15 | Nucleotide sequence of FAD2-T2 probe |
| SEQ ID NO: 16 | Nucleotide sequence of FAD3-F1 primer |
| SEQ ID NO: 17 | Nucleotide sequence of FAD3-R2 primer |
| SEQ ID NO: 18 | Nucleotide sequence of FAD3-T2 probe |
| SEQ ID NO: 19 | Nucleotide sequence of FAD3-F2 primer |
| SEQ ID NO: 20 | Nucleotide sequence of SIP-130F primer |
| SEQ ID NO: 21 | Nucleotide sequence of SIP-198R primer |
| SEQ ID NO: 22 | Nucleotide sequence of SIP-170T probe |
| SEQ ID NO: 23 | Nucleotide sequence of PCR primer WOL1007 |
| SEQ ID NO: 24 | Nucleotide sequence of PCR primer WOL1008 |
| SEQ ID NO: 25 | Nucleotide sequence of PCR primer WOL1009 |
| SEQ ID NO: 26 | Nucleotide sequence of PCR primer WOL1100 |
| SEQ ID NO: 27 | Nucleotide sequence of PCR primer WOL1101 |
| SEQ ID NO: 28 | Nucleotide sequence of PCR primer WOL1102 |
| SEQ ID NO: 29 | Nucleotide sequence of PCR primer WOL1103 |
| SEQ ID NO: 30 | Nucleotide sequence of RV005393 construct |
| SEQ ID NO: 31 | Nucleotide sequence of RV019928 construct |
| SEQ ID NO: 32 | Nucleotide sequence of RV019930 construct |
| SEQ ID NO: 33 | Nucleotide sequence of FAD2-1A WT allele near GM-FAD2-1 |
| | CR1 site RV019927 experiment |
| SEQ ID NO: 34 | Nucleotide sequence of FAD2-1A edited allele of the 1.1 variant |
| | near GM-FAD2-1 CR1 site RV019927 experiment |
| SEQ ID NO: 35 | Nucleotide sequence of FAD2-1A edited allele of the 1.2 variant |
| | near GM-FAD2-1 CR1 site RV019927 experiment |
| SEQ ID NO: 36 | Nucleotide sequence of FAD2-1A edited allele of the 1.3 variant |
| | near GM-FAD2-1 CR1 site RV019927 experiment |
| SEQ ID NO: 37 | Nucleotide sequence of FAD2-1A edited allele of the 1.4 variant |
| | near GM-FAD2-1 CR1 site RV019927 experiment |
| SEQ ID NO: 38 | Nucleotide sequence of FAD2-1A edited allele of the 1.5 variant |
| | near GM-FAD2-1 CR1 site RV019927 experiment |
| SEQ ID NO: 39 | Nucleotide sequence of FAD2-1A edited allele of the 1.6 variant |
| | near GM-FAD2-1 CR1 site RV019927 experiment |
| SEQ ID NO: 40 | Nucleotide sequence of FAD2-1A edited allele of the 1.7 variant |
| | near GM-FAD2-1 CR1 site RV019927 experiment |
| SEQ ID NO: 41 | Nucleotide sequence of FAD2-1A edited allele of the 1.8 variant |
| | near GM-FAD2-1 CR1 site RV019927 experiment |
| SEQ ID NO: 42 | Nucleotide sequence of FAD2-1B WT allele near GM-FAD2-1 |
| | CR1 site RV019927 experiment |
| SEQ ID NO: 43 | Nucleotide sequence of FAD2-1B edited allele of the 1.1 variant |
| | near GM-FAD2-1 CR1 site RV019927 experiment |
| SEQ ID NO: 44 | Nucleotide sequence of FAD2-1B edited allele of the 1.2 variant |
| | near GM-FAD2-1 CR1 site RV019927 experiment |
| SEQ ID NO: 45 | Nucleotide sequence of FAD2-1B edited allele of the 1.3 variant |
| | near GM-FAD2-1 CR1 site RV019927 experiment |
| SEQ ID NO: 46 | Nucleotide sequence of FAD2-1B edited allele of the 1.4 variant |
| | near GM-FAD2-1 CR1 site RV019927 experiment |
| SEQ ID NO: 47 | Nucleotide sequence of FAD2-1B edited allele of the 1.5 variant |
| | near GM-FAD2-1 CR1 site RV019927 experiment |
| SEQ ID NO: 48 | Nucleotide sequence of FAD2-1B edited allele of the 1.6 variant |
| | near GM-FAD2-1 CR1 site RV019927 experiment |
| SEQ ID NO: 49 | Nucleotide sequence of FAD2-1B edited allele of the 1.7 variant |
| | near GM-FAD2-1 CR1 site RV019927 experiment |
| SEQ ID NO: 50 | Nucleotide sequence of FAD2-1B edited allele of the 1.8 variant |
| | near GM-FAD2-1 CR1 site RV019927 experiment |
| SEQ ID NO: 51 | Nucleotide sequence of FAD2-1A WT allele near GM-FAD2-1 |
| | CR1 site RV019929 experiment |
| SEQ ID NO: 52 | Nucleotide sequence of FAD2-1A edited allele of the 1.1 variant |
| | near GM-FAD2-1 CR1 site RV019929 experiment |
| SEQ ID NO: 53 | Nucleotide sequence of FAD2-1A edited allele of the 1.2 variant |
| | near GM-FAD2-1 CR1 site RV019929 experiment |
| SEQ ID NO: 54 | Nucleotide sequence of FAD2-1A edited allele of the 1.3 variant |
| | near GM-FAD2-1 CR1 site RV019929 experiment |
| SEQ ID NO: 55 | Nucleotide sequence of FAD2-1A edited allele of the 1.4 variant |
| | near GM-FAD2-1 CR1 site RV019929 experiment |
| SEQ ID NO: 56 | Nucleotide sequence of FAD2-1A edited allele of the 1.5 variant |
| | near GM-FAD2-1 CR1 site RV019929 experiment |
| SEQ ID NO: 57 | Nucleotide sequence of FAD2-1A edited allele of the 1.6 variant |
| | near GM-FAD2-1 CR1 site RV019929 experiment |
| SEQ ID NO: 58 | Nucleotide sequence of FAD2-1A edited allele of the 1.7 variant |
| | near GM-FAD2-1 CR1 site RV019929 experiment |
| SEQ ID NO: 59 | Nucleotide sequence of FAD2-1A edited allele of the 1.8 variant |
| | near GM-FAD2-1 CR1 site RV019929 experiment |
| SEQ ID NO: 60 | Nucleotide sequence of FAD2-1B WT allele near GM-FAD2-1 |
| | CR1 site RV019929 experiment |
| SEQ ID NO: 61 | Nucleotide sequence of FAD2-1B edited allele of the 1.1 variant |
| | near GM-FAD2-1 CR1 site RV019929 experiment |
| SEQ ID NO: 62 | Nucleotide sequence of FAD2-1B edited allele of the 1.2 variant |
| | near GM-FAD2-1 CR1 site RV019929 experiment |
| SEQ ID NO: 63 | Nucleotide sequence of FAD2-1B edited allele of the 1.3 variant |
| | near GM-FAD2-1 CR1 site RV019929 experiment |
| SEQ ID NO: 64 | Nucleotide sequence of FAD2-1B edited allele of the 1.4 variant |
| | near GM-FAD2-1 CR1 site RV019929 experiment |
| SEQ ID NO: 65 | Nucleotide sequence of FAD2-1B edited allele of the 1.5 variant |
| | near GM-FAD2-1 CR1 site RV019929 experiment |
| SEQ ID NO: 66 | Nucleotide sequence of FAD2-1B edited allele of the 1.6 variant |
| | near GM-FAD2-1 CR1 site RV019929 experiment |
| SEQ ID NO: 67 | Nucleotide sequence of FAD2-1B edited allele of the 1.7 variant |
| | near GM-FAD2-1 CR1 site RV019929 experiment |
| SEQ ID NO: 68 | Nucleotide sequence of FAD2-1B edited allele of the 1.8 variant |
| | near GM-FAD2-1 CR1 site RV019929 experiment |
| SEQ ID NO: 69 | Nucleotide sequence of FAD3a WT allele near GM-FAD3 CR2 |
| | site RV019929 experiment |
| SEQ ID NO: 70 | Nucleotide sequence of FAD3a edited allele of the 1.1 variant near |
| | GM-FAD3 CR2 site RV019929 experiment |
| SEQ ID NO: 71 | Nucleotide sequence of FAD3a edited allele of the 1.2 variant near |
| | GM-FAD3 CR2 site RV019929 experiment |
| SEQ ID NO: 72 | Nucleotide sequence of FAD3a edited allele of the 1.3 variant near |
| | GM-FAD3 CR2 site RV019929 experiment |
| SEQ ID NO: 73 | Nucleotide sequence of FAD3a edited allele of the 1.4 variant near |
| | GM-FAD3 CR2 site RV019929 experiment |
| SEQ ID NO: 74 | Nucleotide sequence of FAD3a edited allele of the 1.5 variant near |
| | GM-FAD3 CR2 site RV019929 experiment |
| SEQ ID NO: 75 | Nucleotide sequence of FAD3a edited allele of the 1.6 variant near |
| | GM-FAD3 CR2 site RV019929 experiment |
| SEQ ID NO: 76 | Nucleotide sequence of FAD3b WT allele near GM-FAD3 CR2 |
| | site |
| SEQ ID NO: 77 | Nucleotide sequence of FAD3b edited allele of the 1.1 variant near |
| | GM-FAD3 CR2 site RV019929 experiment |
| SEQ ID NO: 78 | Nucleotide sequence of FAD3b edited allele of the 1.2 variant near |
| | GM-FAD3 CR2 site RV019929 experiment |
| SEQ ID NO: 79 | Nucleotide sequence of FAD3b edited allele of the 1.3 variant near |
| | GM-FAD3 CR2 site RV019929 experiment |
| SEQ ID NO: 80 | Nucleotide sequence of FAD3b edited allele of the 1.4 variant near |
| | GM-FAD3 CR2 site RV019929 experiment |
| SEQ ID NO: 81 | Nucleotide sequence of FAD3b edited allele of the 1.5 variant near |
| | GM-FAD3 CR2 site RV019929 experiment |
| SEQ ID NO: 82 | Nucleotide sequence of FAD3b edited allele of the 1.6 variant near |
| | GM-FAD3 CR2 site RV019929 experiment |
| SEQ ID NO: 83 | Nucleotide sequence of FAD3b edited allele of the 1.7 variant near |
| | GM-FAD3 CR2 site RV019929 experiment |
| SEQ ID NO: 84 | Nucleotide sequence of FAD3b edited allele of the 1.8 variant near |
| | GM-FAD3 CR2 site RV019929 experiment |
| SEQ ID NO: 85 | Nucleotide sequence of soybean FAD2-1A gene. |
| SEQ ID NO: 86 | Nucleotide sequence of soybean FAD2-1B gene |
| SEQ ID NO: 87 | Nucleotide sequence of soybean FAD3a gene |
| SEQ ID NO: 88 | Nucleotide sequence of soybean FAD3b gene |
| SEQ ID NO: 89 | Nucleotide sequence of soybean FAD2-1A coding sequence (CDS) |
| SEQ ID NO: 90 | Amino acid sequence of soybean FAD2-1A gene |
| SEQ ID NO: 91 | Nucleotide sequence of soybean FAD2-1B coding sequence (CDS) |
| SEQ ID NO: 92 | Amino acid sequence of soybean FAD2-1B gene |
| SEQ ID NO: 93 | Nucleotide sequence of soybean FAD3a coding sequence (CDS) |
| SEQ ID NO: 94 | Amino acid sequence of soybean FAD3a gene |
| SEQ ID NO: 95 | Nucleotide sequence of soybean FAD3b coding sequence (CDS) |
| SEQ ID NO: 96 | Amino acid sequence of soybean FAD3b gene |
| SEQ ID NO: 97 | Nucleotide sequence of Vector 9 |
| SEQ ID NO: 98 | Nucleotide sequence of Vector 10 |
| SEQ ID NO: 99 | Nucleotide sequence of Vector 11 |
| SEQ ID NO: 100 | Nucleotide sequence of Vector 12 |
| SEQ ID NO: 101 | Nucleotide sequence of Vector 13 |
| SEQ ID NO: 102 | Nucleotide sequence of Vector 14 |
| SEQ ID NO: 103 | Nucleotide sequence of Vector 15 |
| SEQ ID NO: 104 | Nucleotide sequence of Vector 16 |
| SEQ ID NO: 105 | Nucleotide sequence of Vector 17 |
| SEQ ID NO: 106 | Artificial DNA sequence depicted in FIG. 19B for the WT soy |
| | sequence (SEQ ID NO: 106) |
| SEQ ID NO: 107 | Artificial DNA sequence depicted in FIG. 19B for the donor DNA |
| | for gRNA2 (SEQ ID NO: 107) |
| SEQ ID NO: 108 | Nucleotide sequence of PHP70365; pVIR8 |

In an aspect, compositions and methods related to improving the editing of endogenous and previously-introduced heterologous polynucleotides and for improving the frequency and efficiency of homology-directed repair of double-strand-break sites are provided. In an aspect, plants, such as soybean plants, have been modified using genomic editing techniques disclosed herein to provide an improved editing efficiency of endogenous and previously-introduced heterologous polynucleotides and an improved frequency and efficiency of homology-directed repair of double-strand-break sites are provided. In an aspect, the present disclosure found that modifying targeted DNA breaks at genomic loci of a plant using genomic editing technology, including the editing T-DNA as described herein provided improved editing of endogenous and previously-introduced heterologous polynucleotides and improved the frequency and efficiency of homology-directed repair of double-strand-break sites. In an aspect, the editing T-DNA comprises a gRNA cassette near the right border and placed upstream of a Cas endonuclease expression cassette in a binary vector transformation system. In an aspect, the binary vector transformation system comprising the editing T-DNA is delivered to a soybean plant cell by Ochrobactrum-mediated transformation.

In an aspect, modified seeds, such as soybean seeds, are provided with increased levels of oleic acid and decreased levels of linolenic acid. The soybeans described herein may further contain one or more of decreased levels of saturated fatty acids, such as one or more of palmitic and stearic acids, and decreased levels of linoleic acid.

Oils produced from seeds described herein may contain low levels of saturated fatty acids which are desirable in providing a healthy diet. Fats that are solid at room temperature can be used in applications such as the production of non-dairy margarines and spreads, and various applications in confections and in baking. Provided are oils and triglycerides for solid fat applications which may contain a predominance of the very high melting, long chain fatty acid stearic acid and a balance of monounsaturated fatty acid with very little polyunsaturated fat. Solid fat fractions having a triacylglyceride structure with saturated fatty acids occupying the sn-1 and sn-3 positions of the triglycerides and an unsaturated fatty acid at the sn-2 position are provided. This overall fatty acid composition and triglyceride structure confers an optimal solid fat crystal structure and a maximum melting point with minimal saturated fatty acid content.

The modified plants, seeds and oil compositions disclosed herein are produced by genomic editing techniques which facilitate the improved editing of the FAD2-1A, FAD-2-1B, FAD3a and FAD3b alleles. The sense strand or the complement thereof may be edited.

A “FAD2”, “FAD2-1”, “FAD2-1A” or FAD2-1B″ or a “FAD2-modified plant”, “FAD2-1-modified plant”, “FAD2-1A-modified plant” or “FAD2-1B modified plant” generally refers to a modified plant or mutant plant that has one or more nucleotide changes in a genomic region that encodes a FAD2, FAD2-1, FAD2-1A or FAD2-1B polypeptide. A “FAD3”, “FAD3a”, “FAD3b”, “FAD3-modified plant”, “FAD3a-modified plant” or a “FAD3b-modified plant generally refers to a modified plant or mutant plant that has one or more nucleotide changes in a genomic region that encodes a FAD3, FAD3a, FAD3b polypeptide. The nucleotide changes in the genomic regions of FAD2, FAD2-1, FAD2-1A or FAD2-1B and/or FAD3, FAD3a, FAD3b can include modifications that result in one or more of SEQ ID NOs: 34-41, 43-50, 52-59, 61-68, 70-75 and 77-84 being contained within the genomic region.

In some aspects, the polynucleotides disclosed herein may be isolated polynucleotides. An “isolated polynucleotide” generally refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A regulatory element generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located. The nucleic acid molecule regulated by a regulatory element does not necessarily have to encode a functional peptide or polypeptide, e.g., the regulatory element can modulate the expression of a short interfering RNA or an anti-sense RNA.

An enhancer element is any nucleic acid molecule that increases transcription of a nucleic acid molecule when functionally linked to a promoter regardless of its relative position. An enhancer may be an innate element of the promoter or a heterologous element inserted to enhance the level or tissue-specificity of a promoter.

A repressor (also sometimes called herein silencer) is defined as any nucleic acid molecule which inhibits the transcription when functionally linked to a promoter regardless of relative position.

Promotors which may be useful in the methods and compositions provided include those containing cis elements, promoters functional in a plant cell, tissue specific and tissue-preferred promotors, developmentally regulated promoters and constitutive promoters. “Promoter” generally refers to a nucleic acid fragment capable of controlling transcription of another nucleic acid fragment. A promoter generally includes a core promoter (also known as minimal promoter) sequence that includes a minimal regulatory region to initiate transcription, that is a transcription start site.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

“Promoter functional in a plant” is a promoter capable of initiating transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” generally refers to a promoter whose activity is determined by developmental events.

“Constitutive promoter” generally refers to promoters active in all or most tissues or cell types of a plant at all or most developing stages. As with other promoters classified as “constitutive” (e.g. ubiquitin), some variation in absolute levels of expression can exist among different tissues or stages. The term “constitutive promoter” or “tissue-independent” are used interchangeably herein.

Provided are sequences which are heterologous nucleotide sequences which can be used in the methods and compositions disclosed herein. A “heterologous nucleotide sequence” generally refers to a sequence that is not naturally occurring with the sequence of the disclosure. While this nucleotide sequence is heterologous to the sequence, it may be homologous, or native, or heterologous, or foreign, to the plant host. However, it is recognized that the instant sequences may be used with their native coding sequences to increase or decrease expression resulting in a change in phenotype in the transformed seed. The terms “heterologous nucleotide sequence”, “heterologous sequence”, “heterologous nucleic acid fragment”, and “heterologous nucleic acid sequence” are used interchangeably herein.

The polynucleotide sequence of the targets of the present disclosure (e.g., SEQ ID NOS: 85-88) and the coding sequences SEQ ID NO: 89, 91, 93, or 95, encoding the polypeptides 90, 92, 94 or 96 respectively, may be modified or altered to reduce their expression or the characteristics of the protein. Examples of such modifications are one or more of the sequences listed in Table 1. As one of ordinary skill in the art will appreciate, modification or alteration can also be made without substantially affecting the gene expression function. The methods are well known to those of skill in the art. Sequences can be modified, for example by insertion, deletion, or replacement of template sequences through any modification approach. The genomic sequences contain introns and exons which may be targeted according to the methods disclosed herein.

SEQ ID NO: 85 (soybean FAD2-1A gene) has the start codon at position 1-3 and the stop codon at position 1329-1331, exon1 is from positions 1-3, intron1 is from positions 4-170, exon2 is from positions 171-1331.

SEQ ID NO: 86 (soybean FAD2-1B gene) has the start codon at position 1-3 and the stop codon at position 1322-1324, exon1 is from positions 1-3, intron1 is from positions 4-163, exon2 is from positions 164-1324.

SEQ ID NO: 87 (soybean FAD3a gene) has the start codon at position 1-3 and the stop codon at position 3866-3868, exon1 is from positions 1-293, intron1 is from positions 294-460, exon2 is from positions 461-550, intron2 is from positions 551-874, exon3 is from positions 875-941, intron3 is from positions 942-1076, exon4 is from positions 1077-1169, intron4 is from positions 1170-1278, exon5 is from positions 1279-1464, intron5 is from positions 1465-1756, exon6 is from positions 1757-1837, intron6 is from positions 1838-2874, exon7 is from positions 2875-3012, intron1 is from positions 3013-3685, exon8 is from positions 3686-3868.

SEQ ID NO: 88 (soybean FAD3b gene) has the start codon at position 1-3 and the stop codon at position 3894-3896, exon1 is from positions 1-305, intron1 is from positions 306-497, exon2 is from positions 498-587, intron2 is from positions 588-935, exon3 is from positions 936-1002, intron3 is from positions 1003-1144, exon4 is from positions 1145-1237, intron4 is from positions 1238-1335, exon5 is from positions 1336-1521, intron5 is from positions 1522-1636, exon6 is from positions 1637-1717, intron6 is from positions 1637-1717, exon7 is from positions 2950-3087, intron7 is from positions 3088-3713, exon8 is from positions 3714-3896.

Variant promotors can be used in the methods and compositions disclosed herein. A “variant promoter” as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Provided are sequences that are a full complement or a full-length complement of those disclosed herein, such as the nucleotide sequences in Table 1. The terms “full complement” and “full-length complement” are used interchangeably herein, and refer to a complement of a given nucleotide sequence, wherein the complement and the nucleotide sequence consist of the same number of nucleotides and are 100% complementary.

Provided are sequences that are “substantially similar” or “corresponding substantially” to those disclosed herein which can be used in the methods and compositions described herein. The terms “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences.

Provided are compositions and methods that includes materials, steps, features, components, or elements that consist essentially of a particular component. The transitional phrase “consisting essentially of” generally refers to a composition, method that includes materials, steps, features, components, or elements, in addition to those literally disclosed, provided that these additional materials, steps, features, components, or elements do not materially affect the basic and novel characteristic(s) of the claimed subject matter, e.g., one or more of the claimed sequences.

Moreover, the skilled artisan recognizes that substantially similar nucleic acid sequences encompassed by this disclosure are also defined by their ability to hybridize, under moderately stringent conditions (for example, 0.5×SSC, 0.1% SDS, 60° C.) with the sequences exemplified herein, or to any portion of the nucleotide sequences reported herein and which are functionally equivalent to the promoter of the disclosure. Estimates of such homology are provided by either DNA-DNA or DNA-RNA hybridization under conditions of stringency as is well understood by those skilled in the art (Hames and Higgins, Eds.; In Nucleic Acid Hybridisation; IRL Press: Oxford, U. K., 1985). Stringency conditions can be adjusted to screen for moderately similar fragments, such as homologous sequences from distantly related organisms, to highly similar fragments, such as genes that duplicate functional enzymes from closely related organisms. Post-hybridization washes partially determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Another set of stringent conditions uses higher temperatures in which the washes are identical to those above except for the temperature of the final two 30 min washes in 0.2×SSC, 0.5% SDS was increased to 60° C. Another set of highly stringent conditions uses two final washes in 0.1×SSC, 0.1% SDS at 65° C.

Provided are substantially similar sequences useful in compositions and methods provided herein. A “substantially similar sequence” generally refers to variants of the disclosed sequences such as those that result from site-directed mutagenesis, as well as synthetically derived sequences. Sequence alignments and percent identity calculations may be determined using a variety of comparison methods designed to detect similar or identical sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

In an aspect, the % sequence identity is determined over the entire length of the molecule (nucleotide or amino acid). A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN generally refers to a BLAST program that compares a nucleotide query sequence against a nucleotide sequence database.

The present disclosure provides genes, mutated genes, chimeric genes and recombinant expression constructs. “Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” generally refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

“Chimeric gene” or “recombinant expression construct”, which are used interchangeably, includes any gene that is not a native gene, comprising regulatory and coding sequences that are not found together in nature. Accordingly, a chimeric gene may comprise regulatory sequences and coding sequences that are derived from different sources.

“Coding sequence” generally refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

The term “operably linked” or “functionally linked” generally refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “initiate transcription”, “initiate expression”, “drive transcription”, and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “expression cassette” as used herein, generally refers to a discrete nucleic acid fragment into which a nucleic acid sequence or fragment can be cloned or synthesized through molecular biology techniques.

As stated herein, “suppression” includes a reduction of the level of enzyme activity or protein functionality (e.g., a phenotype associated with a protein) detectable in a transgenic plant when compared to the level of enzyme activity or protein functionality detectable in a non-transgenic or wild type plant with the native enzyme or protein. The level of enzyme activity in a plant with the native enzyme is referred to herein as “wild type” activity. The level of protein functionality in a plant with the native protein is referred to herein as “wild type” functionality. The term “suppression” includes lower, reduce, decline, decrease, inhibit, eliminate and prevent. This reduction may be due to a decrease in translation of the native mRNA into an active enzyme or functional protein. It may also be due to the transcription of the native DNA into decreased amounts of mRNA and/or to rapid degradation of the native mRNA. The term “native enzyme” generally refers to an enzyme that is produced naturally in a non-transgenic or wild type cell. The terms “non-transgenic” and “wild type” are used interchangeably herein.

“Altering expression” or “modulating expression” generally refers to the production of gene product(s) in plants in amounts or proportions that differ significantly from the amount of the gene product(s) produced by the corresponding wild-type plants (i.e., expression is increased or decreased).

“Transformation” as used herein generally refers to both stable transformation and transient transformation. In an aspect, bacterial strains useful in the methods for improving editing of endogenous and previously-introduced heterologous polynucleotides and for improving the frequency and efficiency of homology-directed repair of double-strand-break sites of the disclosure include, but are not limited to, a disarmed Agrobacteria, an Ochrobactrum bacteria or a Rhizobiaceae bacteria. In an aspect, the different bacterial strains are selected from (i) a disarmed Agrobacteria and an Ochrobactrum bacteria, (ii) a disarmed Agrobacteria and a Rhizobiaceae bacteria, and (iii) a Rhizobiaceae bacteria and an Ochrobactrum bacteria.

In an aspect, disarmed Agrobacteria useful in the present methods include, but are not limited to, AGL-1, EHA105, GV3101, LBA4404, and LBA4404 THY-.

In an aspect, Ochrobactrum bacterial strains useful in the present methods for improving editing of endogenous and previously-introduced heterologous polynucleotides and for improving the frequency and efficiency of homology-directed repair of double-strand-break sites include, but are not limited to, Ochrobactrum haywardense H1 NRRL Deposit B-67078, Ochrobactrum cytisi, Ochrobactrum daejeonense, Ochrobactrum oryzae, Ochrobactrum tritici LBNL124-A-10, HTG3-C-07, Ochrobactrum pecoris, Ochrobactrum ciceri, Ochrobactrum gallinifaecis, Ochrobactrum grignonense, Ochrobactrum guangzhouense, Ochrobactrum haematophilum, Ochrobactrum intermedium, Ochrobactrum lupini, Ochrobactrum pituitosum, Ochrobactrum pseudintermedium, Ochrobactrum pseudogrignonense, Ochrobactrum rhizosphaerae, Ochrobactrum thiophenivorans, and Ochrobactrum tritici disclosed in US Patent Publication No. 20180216123 incorporated herein by reference in its entirety.

In an aspect, Rhizobiaceae bacterial strains useful in the present methods for improving editing of endogenous and previously-introduced heterologous polynucleotides and for improving the frequency and efficiency of homology-directed repair of double-strand-break sites include, but are not limited to, Rhizobium lusitanum, Rhizobium rhizogenes, Agrobacterium rubi, Rhizobium multihospitium, Rhizobium tropici, Rhizobium miluonense, Rhizobium leguminosarum, Rhizobium leguminosarum bv. trifolii, Rhizobium leguminosarum bv. phaseoli, Rhizobium leguminosarum. bv. viciae, Rhizobium leguminosarum Madison, Rhizobium leguminosarum USDA2370, Rhizobium leguminosarum USDA2408, Rhizobium leguminosarum USDA2668, Rhizobium leguminosarum 2370G, Rhizobium leguminosarum 2370LBA, Rhizobium leguminosarum 2048G, Rhizobium leguminosarum 2048LBA, Rhizobium leguminosarum bv. phaseoli 2668G, Rhizobium leguminosarum bv. phaseoli 2668LBA, Rhizobium leguminosarum RL542C, Rhizobium etli USDA 9032, Rhizobium etli bv. phaseoli, Rhizobium endophyticum, Rhizobium tibeticum, Rhizobium etli, Rhizobium pisi, Rhizobium phaseoli, Rhizobium fabae, Rhizobium hainanense, Arthrobacter viscosus, Rhizobium alamii, Rhizobium mesosinicum, Rhizobium sullae, Rhizobium indigoferae, Rhizobium gallicum, Rhizobium yanglingense, Rhizobium mongolense, Rhizobium oryzae, Rhizobium loessense, Rhizobium tubonense, Rhizobium cellulosilyticum, Rhizobium soli, Neorhizobium galegae, Neorhizobium vignae, Neorhizobium huautlense, Neorhizobium alkalisoli, Aureimonas altamirensis, Aureimonas frigidaquae, Aureimonas ureilytica. Aurantimonas coralicida, Fulvimarina pelagi, Martelella mediterranea, Allorhizobium undicola, Allorhizobium vitis, Allorhizobium bonbon, Beijerinckia fluminensis, Agrobacterium larrymoorei, Agrobacterium radiobacter, Rhizobium selenitireducens corrig. Rhizobium rosettiformans, Rhizobium daejeonense, Rhizobium aggregatum, Pararhizobium capsulatum, Pararhizobium giardinii, Ensifer mexicanus, Ensifer tenangae, Ensifer saheli, Ensifer kostiensis, Ensifer kummerowiae, Ensifer fredii Sinorhizobium americanum, Ensifer arboris, Ensifer garamanticus, Ensifer meliloti, Ensifer numidicus, Ensifer adhaenens, Sinorhizobium sp., Sinorhizobium meliloti SD630, Sinorhizobium meliloti USDA1002, Sinorhizobium fredii USDA205, Sinorhizobium fredii SF542G, Sinorhizobium fredii SF4404, and Sinorhizobium fredii SM542C. See U.S. Pat. No. 9,365,859 incorporated herein by reference in its entirety.

In an aspect, bacteria of the class Alphaproteobacteria, order Rhizobiales useful in the present methods for improving editing of endogenous and previously-introduced heterologous polynucleotides and for improving the frequency and efficiency of homology-directed repair of double-strand-break sites include, but are not limited to, bacteria of the family Rhizobiaceae, genus Rhizobium, Chelatobacter, Sinorhizobium, and unclassified Rhizobiaceae, the family Bartonellaceae, genus Bartonella and unclassified Bartonellaceae, the family Brucellaceae, genus Brucella, Mycoplana, Ochrobactrum, and unclassified Brucellaceae, the family Phyllobacteriaceae, genus Phyllobacterium, Aminobacter, Aquamicrobium, Defluvibacter, Mesorhizobium, Pseudaminobacter, and unclassified Phyllobacteriaceae, the family Methylocystaceae, genus Methylocytis, Albibacter, Methylosinus, Terasakiella, and unclassified Methylocystaceae, the family Beijerinckiaceae, genus Beijerinckia, and unclassified Beijerinckiaceae, and the family Bradyrhizobium, genus Afipia, Blastobacter, Bosea, Nitrobacter, Rhodoblastus, Rhodopseudomonas, and unclassified Bradyrhizobiaceae.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” generally refers to the introduction of a nucleic acid fragment into the nucleus, or DNA-containing organelle, of a host organism resulting in gene expression without genetically stable inheritance.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Genome” as it applies to plant cells encompasses not only chromosomal DNA found within the nucleus, but organelle DNA found within subcellular components (e.g., mitochondrial, plastid) of the cell.

“Genetic modification” generally refers to modification of any nucleic acid sequence or genetic element by insertion, deletion, or substitution of one or more nucleotides in an endogenous nucleotide sequence by genome editing or by insertion of a recombinant nucleic acid, e.g., as part of a vector or construct in any region of the plant genomic DNA by routine transformation techniques. Examples of modification of genetic components include, but are not limited to, promoter regions, 5′ untranslated leaders, introns, genes, 3′ untranslated regions, and other regulatory sequences or sequences that affect transcription or translation of one or more nucleic acid sequences.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

Provided are plants which are dicots. The terms “dicot” and “dicotyledonous plant” are used interchangeably herein. A dicot of the current disclosure includes the following families: Brassicaceae, Leguminosae, and Solanaceae.

In an aspect, the methods of the present disclosure may be used for increasing the efficiency of editing of endogenous and previously-introduced heterologous polynucleotides and for improving the frequency and efficiency of homology-directed repair of double-strand-break sites of any plant species, including, but not limited to, monocots and dicots including, but not limited to maize, alfalfa, sorghum, rice, millet, soybean, wheat, cotton, sunflower, barley, oats, rye, flax, sugarcane, banana, cassava, common bean, cowpea, tomato, potato, beet, grape, Eucalyptus, poplar, pine, douglas fir, citrus, papaya, cacao, cucumber, apple, Capsicum, bamboo, Triticale, melon, and Brassica. In an aspect, monocots include, but are not limited to, barley, maize (corn), millet (e.g., pearl millet (Pennisetum glaucum), proso millet (Panicum miliaceum), foxtail millet (Setaria italica), finger millet (Eleusine coracana)), oats, rice, rye, Setaria sp., sorghum, triticale, or wheat, or leaf and stem crops, including, but not limited to, bamboo, marram grass, meadow-grass, reeds, ryegrass, sugarcane; lawn grasses, ornamental grasses, and other grasses such as switchgrass and turf grass. Alternatively, dicot plants used in the present disclosure, include, but are not limited to, kale, cauliflower, broccoli, mustard plant, cabbage, pea, clover, alfalfa, broad bean, tomato, peanut, cassava, soybean, canola, sunflower, safflower, tobacco, Arabidopsis, or cotton.

In specific aspects, plants in which the methods of the present disclosure are useful for increasing the efficiency of editing of endogenous and previously-introduced heterologous polynucleotides and for improving the frequency and efficiency of homology-directed repair of double-strand-break sites are crop plants (for example, corn, alfalfa, sunflower, Brassica, soybean, cotton, safflower, peanut, rice. sorghum, wheat, millet, tobacco, etc.).

Progeny plants are provided. “Progeny” comprises any subsequent generation of a plant, and can include F1 progeny, F2 progeny F3 progeny and so on.

Various changes in phenotype are of interest including, but not limited to, modifying the fatty acid composition in a plant, seed or oil extracted therefrom, altering the fatty acid profile of a plant seed, altering the amounts of fatty acids in a plant seed on seed oil, and the like as disclosed herein. Plants having a desirable phenotype and seed and oil compositions having a fatty acid profile as disclosed herein can be generated by modulating the suppression of FAD2 and FAD3, for example by modulating the suppression of multiple FAD2 and FAD3 alleles, such as one, two, three or all of FAD2-1A, FAD2-1B, FAD-3a and FAD3b alleles. Target sites within the FAD2 and FAD3 alleles can be used to generate short deletions and modifications such as described in Table 1. In an embodiment, the plants and seeds modified as disclosed herein, contain only modified genomic sequence, with no heterologous or foreign DNA remaining in the plant from the modification or in the genomic region of the modification or at the target site. Examples of target sites in soybean include GM-FAD2-1 CR1 (SEQ ID NO: 7) at Gm10:50014185 . . . 50014166 and Gm20:35317773 . . . 35317754 and GM-FAD3 CR2 (SEQ ID NO: 8) at Gm14:45939600 . . . 445939618 and Gm02:41423563 . . . 41423581.

Provided are seeds, such as soybean seeds, which can be processed to produce oils, and the oils produced therefrom, which contain any combination of oleic acid, linolenic acid, linoleic acid, erucic acid (C:22:1) and saturated fatty acids such as stearic acid and palmitic acid in the amounts disclosed herein. Other saturated fatty acids in the soybean seeds and oils which may be increased or decreased compared with a control plant, seed or oil include myristic acid (C:14:0), and long chain saturated fatty acids arachidic acid (C20:0), behenic acid (C22:0) and lignoceric acid (C24:0).

Provided are seeds, such as soybean seeds, which can be processed to produce oils, and the oils produced therefrom, which have at least or at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 percent oleic (C 18:1) acid of the total fatty acids by weight and less than or less than about 100, 99, 98, 97, 96, 95, 94, 93, 92, 91, 90, 89, 88, 87, 86, 85, 84, 83, 82, 81, 80, 79, 78, 76, 75, 74, 73, 72, 71 or 70 percent oleic acid of the total fatty acids by weight.

Provided are seeds, such as soybean seeds, which can be processed to produce oils, and the oils produced therefrom, which have at least or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 percent linolenic (C 18:3) acid of the total fatty acids by weight and less than or less than about 6, 5.5, 5, 4.5, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1 or 2.0 percent linolenic acid of the total fatty acids by weight.

Provided are soybean seeds which can be processed to produce oils, and the oils produced therefrom, which have at least or at least about 0.5, 1, 2, 3, 4, 5, 6, or 7 percent linoleic (C 18:2) acid of the total fatty acids by weight and less than or less than about 55, 50, 45, 40, 35, 30, 25, 20, 15, 10, 9, 8, 7, 6, 5, 4, or 3 percent linoleic acid of the total fatty acids by weight.

Provided are seeds, such as soybean seeds, which can be processed to produce oils, and the oils produced therefrom, which have at least or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 percent stearic acid (C 18:0) of the total fatty acids by weight and less than or less than about 6, 5.5, 5, 4.5, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1 or 2.0 percent stearic acid of the total fatty acids by weight. Provided are seeds, such as soybean seeds, which can be processed to produce oils, and the oils produced therefrom, which have at least or at least about 0.1, 0.5, 1.0, 1.5, 2.0, 2.5, 3.0, 3.5, 4.0, 4.5, 5.0, 5.5, 6.0, 6.5 or 7.0 percent palmitic acid (C 16:0) of the total fatty acids by weight and less than or less than about 12, 11.5, 11.0, 10.5, 10.0, 9.5, 9.0, 8.5, 8.0, 7.5, 7.0, 6.5, 6.0, 5.5, 5.0, 4.5, 4.0, 3.5, 3.0, 2.5, 2.0, 1.5, or 1.0 percent palmitic acid of the total fatty acids by weight.

Provided are seeds, such as soybean seeds, which can be processed to produce oils, and the oils produced therefrom, which have at least or at least about 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, or 11.0 percent total saturated fatty acids of the total fatty acids by weight and less than or less than about 16, 15.5, 15, 14.5, 14, 13.5, 13.0, 12.5, 12.0, 11.5, 11.0, 10.5, 10.0, 9.5, 9.0, 8.5, 8.0, 7.5, 7.0, 6.5, 6.0, 5.5, 5.0, 4.5, 4.0, 3.5, 3.0, 2.5 or 2.0 percent total saturated fatty acids of the total fatty acids by weight.

In an aspect, the seeds, such as soybean seeds, which can be processed to produce oils, and the oils produced therefrom, contain elevated oleic and reduced linolenic acid as described herein, and optionally other modified amounts of other fatty acids as described herein.

In an aspect, this disclosure concerns host cells comprising either the recombinant DNA constructs of the disclosure as described herein or isolated polynucleotides of the disclosure as described herein. Examples of host cells which can be used to practice the disclosure include, but are not limited to, yeast, bacteria, and plants. In an aspect, Ochrobactrum host cells comprising the recombinant DNA constructs of the disclosure as described herein for improving or enhancing editing of endogenous and previously-introduced heterologous polynucleotides and for improving or enhancing the frequency and efficiency of homology-directed repair of double-strand-break sites are provided.

A polynucleotide modification template can be introduced into a cell by any method known in the art, such as, but not limited to, transient introduction methods, transfection, transformation, electroporation, microinjection, particle mediated delivery, topical application, whiskers mediated delivery, delivery via cell-penetrating peptides, or mesoporous silica nanoparticle (MSN)-mediated direct delivery. In an aspect, a polynucleotide modification template cassette is introduced into a cell by Ochrobactrum-mediated transformation.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to transient introduction methods, transfection, microinjection, and/or topical application or indirectly via recombination constructs. The endonuclease can be provided as a protein or as a guided polynucleotide complex directly to a cell or indirectly via recombination constructs. In an aspect, endonuclease comprises and endonuclease cassette and is introduced into a cell by Ochrobactrum-mediated transformation. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in US Patent Publication No. 20170035300, incorporated herein by reference in its entirety.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

Zinc finger nucleases (ZFNs) are engineered double-strand break inducing agents comprised of a zinc finger DNA binding domain and a double-strand-break-inducing agent domain.

Genome editing using DSB-inducing agents, such as Cas9-gRNA complexes, has been described, for example in U.S. Patent Publication Nos. 20150082478 A1, 20150059010 A1, 20170306349 A1 and 20170226533 A1, all of which are incorporated herein by reference in their entireties.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes a Cas9 protein, a Cpf1 protein, a C2c1 protein, a C2c2 protein, a C2c3 protein, Cas3, Cas 5, Cas7, Cas8, Cas10, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Publication No. 20140189896 A1, which is incorporated herein by reference.

Other Cas endonuclease systems have been described in US Patent Publication No. 20180258417 A1, incorporated herein by reference in its entirety.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to, Cas9 and Cpf1 endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide.

In an aspect, the guide polynucleotide can be introduced into a cell transiently, as single stranded polynucleotide or a double stranded polynucleotide, using any method known in the art such as, but not limited to, particle bombardment, Agrobacterium-mediated transformation, Ochrobactrum-mediated transformation, Rhizobiaceae-mediated transformation, or topical applications. In an aspect, the guide polynucleotide can also be introduced indirectly into a cell by introducing a recombinant DNA molecule (via methods such as, but not limited to, particle bombardment, Agrobacterium-mediated transformation, Ochrobactrum-mediated transformation, Rhizobiaceae-mediated transformation, or topical applications) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, published on Feb. 18, 2016, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

Provided are plants and seeds which contain an altered or modified target site or sequence. An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease. Assays to measure the single or double-strand break of a target site by an endonuclease are known in the art and generally measure the overall activity and specificity of the agent on DNA substrates containing recognition sites.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

In an aspect, provided are plants and seeds in which a functional sequence has been knocked out. The terms “knock-out”, “gene knock-out” and “genetic knock-out” are used interchangeably herein. A knock-out represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site.

The guide polynucleotide/Cas endonuclease system can be used in combination with a co-delivered polynucleotide modification template to allow for editing (modification) of a genomic nucleotide sequence of interest. (See, U.S. Patent Publication Nos. 20150082478 A1 and 20150059010 A1, both of which are hereby incorporated herein by reference in their entireties.)

Provided are plants and seeds in which a functional sequence has been knocked in. The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Publication Nos. 20150082478 A1, 20150059010 A1, 20170306349 A1, and 20170226533 A1, all of which are incorporated herein by reference in their entireties) and include, but are not limited to, modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Methods for transforming dicots, by use of Ochrobactrum-mediated transformation disclosed in US Patent Publication No. 20180216123 incorporated herein by reference in its entirety, Rhizobiaceae-mediated transformation (See U.S. Pat. No. 9,365,859 incorporated herein by reference in its entirety), and Agrobacterium-mediated transformation, and obtaining transgenic plants have been published.

There are a variety of methods for the regeneration of plants from plant tissues. The particular method of regeneration will depend on the starting plant tissue and the particular plant species to be regenerated.

In an aspect, this disclosure also concerns methods of increasing the efficiency of editing a target site of a trait of interest or a nucleotide sequence in the genome of a plant which comprise:

(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary to the target site of the trait or nucleotide of interest, a trait or nucleotide of interest modification template cassette, wherein the trait or nucleotide of interest modification template cassette comprises at least one nucleotide modification of the trait or nucleotide of interest, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce a double strand break at the target site of the trait or nucleotide of interest and the trait or nucleotide of interest modification template cassette is capable of making at least one nucleotide modification at the target site of the trait or nucleotide of interest;

(b) identifying at least one plant cell of (a) that has a modification at the target site of the trait or nucleotide of interest, wherein the modification includes at least one deletion or substitution of one or more nucleotides at the target site of the trait or nucleotide of interest; and

(c) regenerating a plant from the at least one plant cell of (b) having the modification at the target site of the trait or nucleotide of interest having increased editing efficiency when compared to a control plant having a modification at the target site of the trait or nucleotide of interest provided by a control plant editing T-DNA, wherein the control plant editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary to the target site and wherein the guide RNA and the Cas endonuclease are capable of forming a complex that enables the Cas endonuclease to introduce cleavage at the target site.

In an aspect, this disclosure also concerns methods of increasing the efficiency of altering the fatty acid profile in the seed of a plant which comprises:

(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, a polynucleotide modification template cassette, wherein the polynucleotide modification template cassette comprises at least one nucleotide modification of a FAD2 genomic sequence in the plant genome, a FAD3 genomic sequence in the plant genome, or a combination thereof, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce a double strand break in the FAD2 genomic sequence in the plant genome, the FAD3 genomic sequence in the plant genome, or a combination thereof and the polynucleotide modification template cassette enables at least one nucleotide modification of the FAD2 genomic sequence in the plant genome, the FAD3 genomic sequence in the plant genome, or the combination thereof;

(b) obtaining a plant from the plant cell of (a);

(c) evaluating the plant of (b) for the presence of the at least one nucleotide modification;

(d) selecting a progeny plant of (c) having an altered fatty acid profile; and

(e) obtaining seed from the progeny plant of (d) having increased editing efficiency when compared to a control seed of a plant having a modification of the FAD2 genomic sequence, the FAD3 genomic sequence, or the combination thereof provided by a control seed editing T-DNA, wherein the control seed editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary to the target site and wherein the guide RNA and the Cas endonuclease are capable of forming a complex that enables the Cas endonuclease to introduce cleavage at the target site. Further screening of a progeny plant of (c) having an altered fatty acid profile may be performed to obtain a progeny plant that is also void of the guide RNA and the Cas endonuclease.

In an aspect, this disclosure also concerns methods for increasing the efficiency of editing a target site in a plant, the method comprising:

(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary to the target site and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage at the target site;

(b) identifying at least one plant cell of (a) that has a modification at the target site, wherein the modification includes at least one deletion or substitution of one or more nucleotides at the target site; and

(c) regenerating a plant from the at least one plant cell of (b) having the modification at the target site having increased editing efficiency when compared to a control plant having a modification at the target site provided by a control plant editing T-DNA, wherein the control plant editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary to the target site and wherein the guide RNA and the Cas endonuclease are capable of forming a complex that enables the Cas endonuclease to introduce cleavage at the target site.

In an aspect, this disclosure also concerns methods of increasing the efficiency of altering the fatty acid profile in the seed of a plant, the method comprising:

(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage in the FAD2 genomic sequence in the plant genome, the FAD3 genomic sequence in the plant genome, or a combination thereof;

(b) obtaining a plant from the plant cell of (a);

(c) evaluating the plant of (b) for the presence of the at least one nucleotide modification;

(d) selecting a progeny plant of (c) having an altered fatty acid profile; and

(e) obtaining seed from the progeny plant of (d) having increased editing efficiency when compared to a control seed of a plant having a modification of the FAD2 genomic sequence, the FAD3 genomic sequence, or the combination thereof provided by a control seed editing T-DNA, wherein the control seed editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof wherein the guide RNA and the Cas endonuclease are capable of forming a complex that enables the Cas endonuclease to introduce cleavage in the FAD2 genomic sequence in the plant genome, the FAD3 genomic sequence in the plant genome, or a combination thereof.

In an aspect, this disclosure also concerns methods of increasing the efficiency of altering the fatty acid profile in the seed of a plant, the method comprising:

(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage within the FAD2 genomic sequence in the plant genome, the FAD3 genomic sequence in the plant genome, or a combination thereof;

(b) obtaining a plant from the plant cell of (a);

(c) evaluating the plant of (b) for the presence of the at least one nucleotide modification;

(d) screening a progeny plant of (c) having an altered fatty acid profile that is void of the guide RNA and the Cas endonuclease; and

(e) obtaining seed from the progeny plant of (d) having increased editing efficiency when compared to a control seed of a plant having a modification of the FAD2 genomic sequence, the FAD3 genomic sequence, or the combination thereof provided by a control seed editing T-DNA, wherein the control seed editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and wherein the guide RNA and the Cas endonuclease are capable of forming a complex that enables the Cas endonuclease to introduce cleavage within the FAD2 genomic sequence in the plant genome, the FAD3 genomic sequence in the plant genome, or a combination thereof.

In an aspect, this disclosure also concerns methods for increasing the efficiency of introducing a nucleotide of interest into a target site in the genome of a plant, the method comprising:

(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to cleavage at the target site;

(b) contacting the plant cell of (a) with a donor DNA comprising the polynucleotide of interest;

(c) identifying at least one plant cell from (b) comprising in its genome the polynucleotide of interest integrated at the target site; and

(d) regenerating a plant from the at least one plant cell having in its genome the polynucleotide of interest integrated at the target site having increased efficiency of introduction of the polynucleotide of interest into the target site in the genome of the plant cell when compared to a control plant having an introduction of the polynucleotide of interest into the target site in the genome of the plant cell provided by a control plant editing T-DNA, wherein the control plant editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA and the Cas endonuclease are capable of forming a complex that enables the Cas endonuclease to cleavage at the target site.

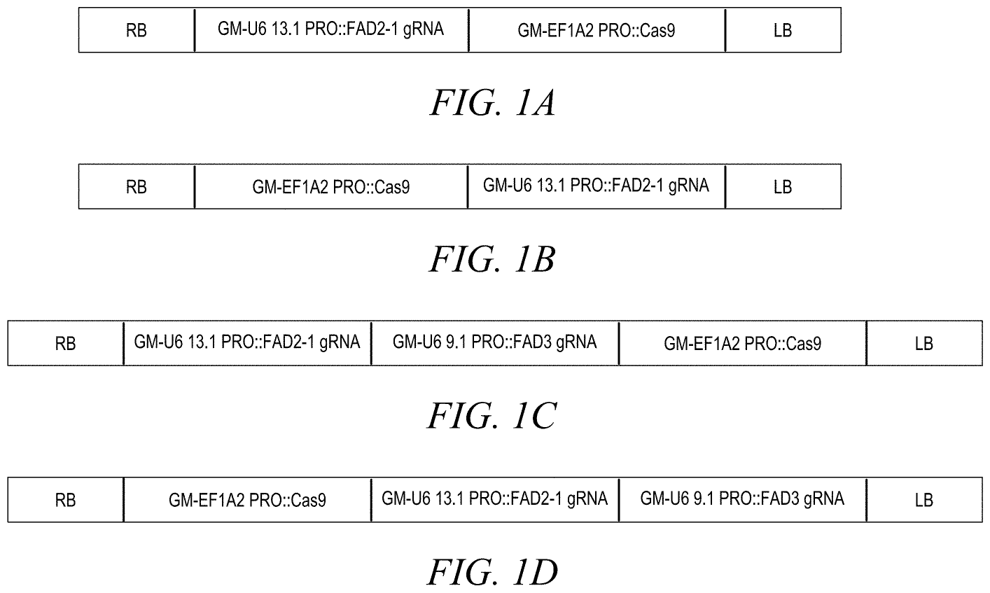

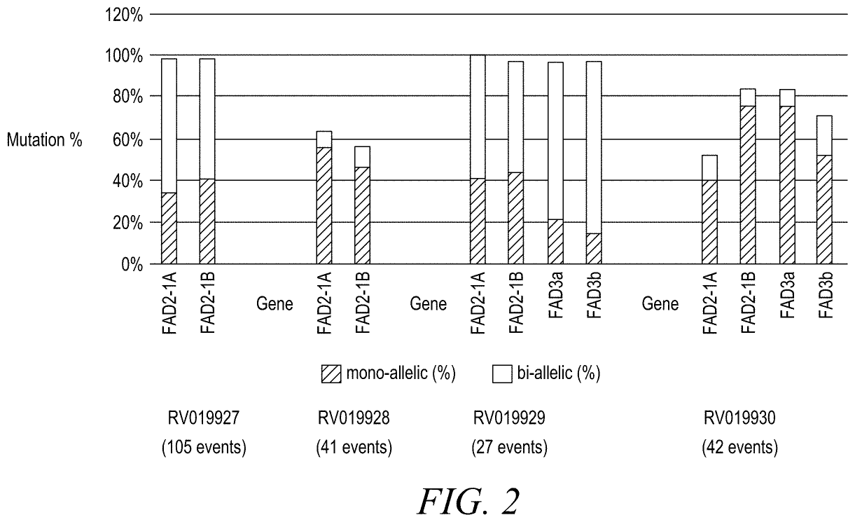

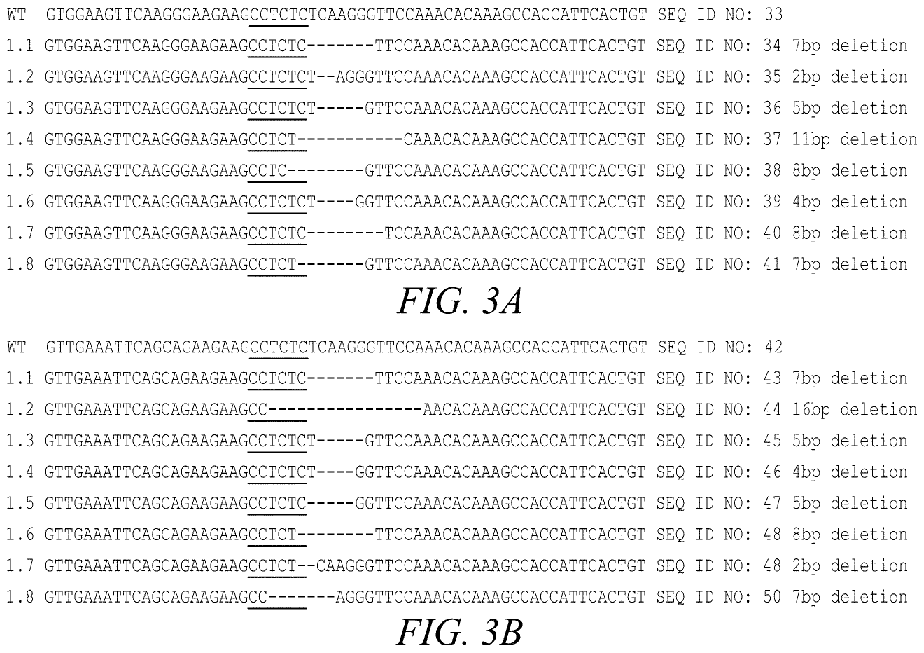

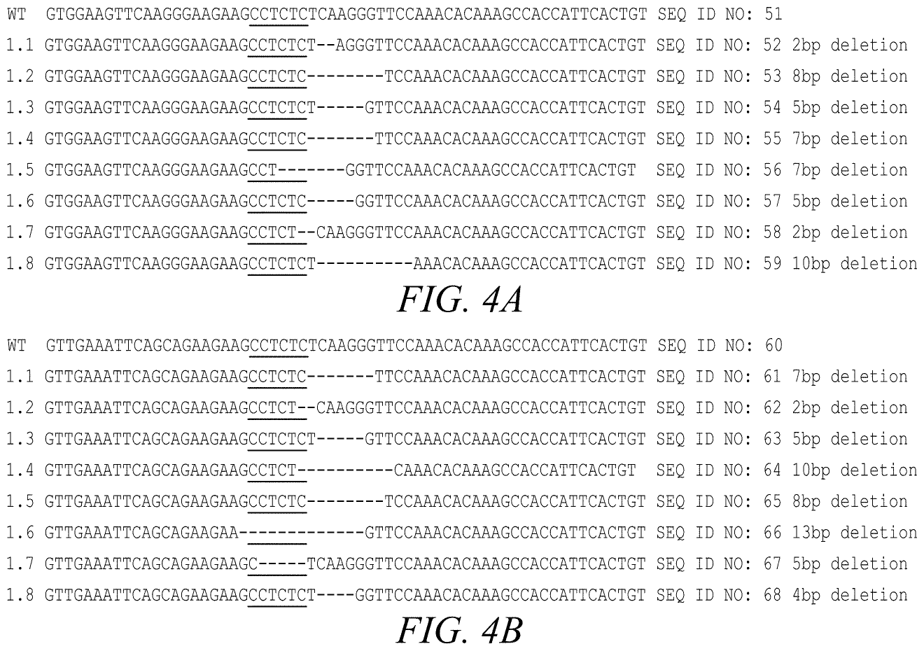

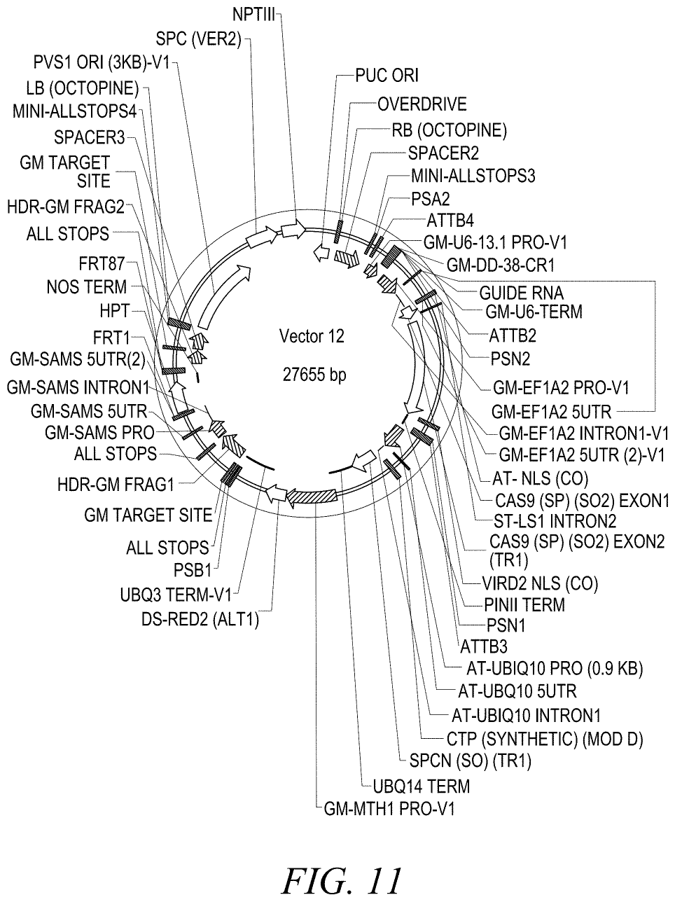

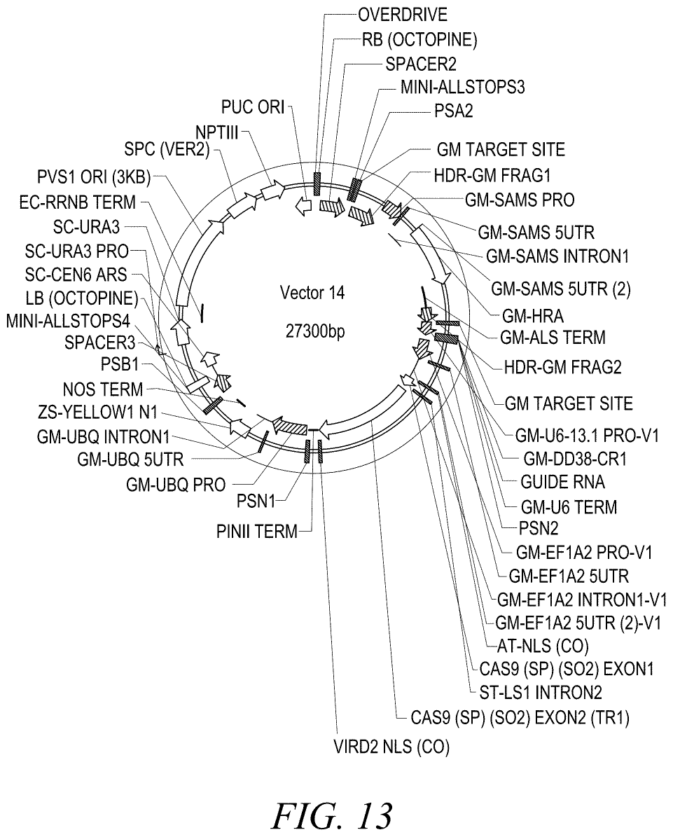

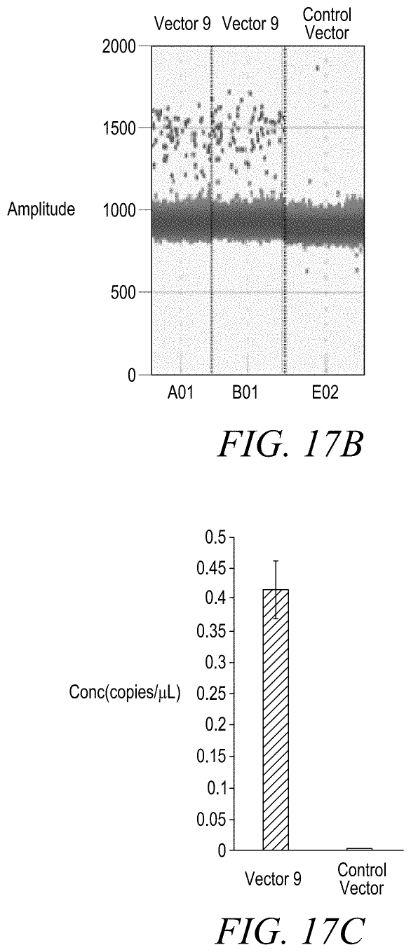

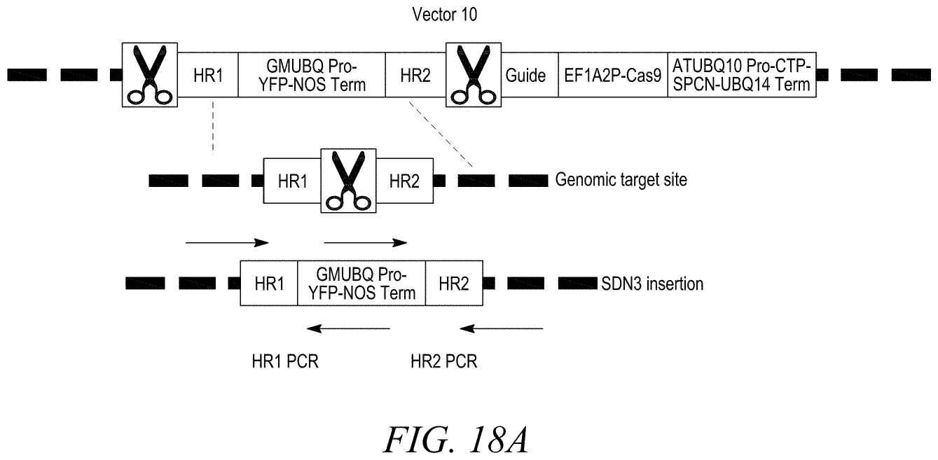

In an aspect, the editing T-DNA comprises in operable linkage from a right border to a left border orientation a guide RNA expression cassette. In an aspect, the guide RNA expression cassette of the editing T-DNA is adjacent to the right border and may be separated from the right border by one or more spacer elements and optionally may be separated from the right border by one or more elements that facilitate PCR analysis. Non-limiting examples of editing T-DNAs useful in the present disclosure are shown in FIG. 8-FIG. 16. See also, FIG. 1A and FIG. 1C.

In an aspect, the control plant editing T-DNA comprises in operable linkage from a left border to a right border orientation a guide RNA expression cassette. In an aspect, the guide RNA expression cassette of the editing T-DNA is adjacent to the left border and may be separated from the left border by one or more spacer elements and optionally may be separated from the right border by one or more elements that facilitate PCR analysis. See FIG. 1B and FIG. 1D.

Transformation and selection can be accomplished using methods well-known to those skilled in the art including, but not limited to, the methods described herein.

The soybean seeds can be processed to produce oil and protein. Methods of processing the soybean seeds to produce oil and protein are provided which include one or more steps of dehulling the seeds, crushing the seeds, heating the seeds, such as with steam, extracting the oil, roasting, and extrusion. Processing and oil extraction can be done using solvents or mechanical extraction.

Products formed following processing include, without limitation, soy nuts, soy milk, tofu, texturized soy protein, soybean oil, soy protein flakes, isolated soy protein. Crude or partially degummed oil can be further processed by one or more of degumming, alkali treatment, silica absorption, vacuum bleaching, hydrogenation, interesterification, filtration, deodorization, physical refining, refractionation, and optional blending to produce refined bleached deodorized (RBD) oil.

The oil and protein can be used in animal feed and in food products for human consumption. Provided are food products and animal feed comprising oils, protein and compositions described herein. The food products and animal feed may comprise nucleotides comprising one or more of the modified alleles disclosed herein.

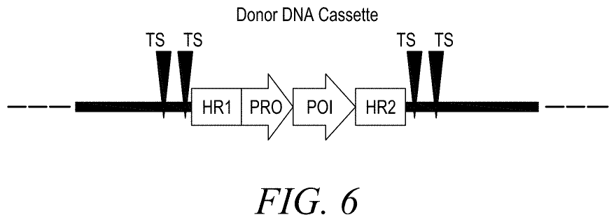

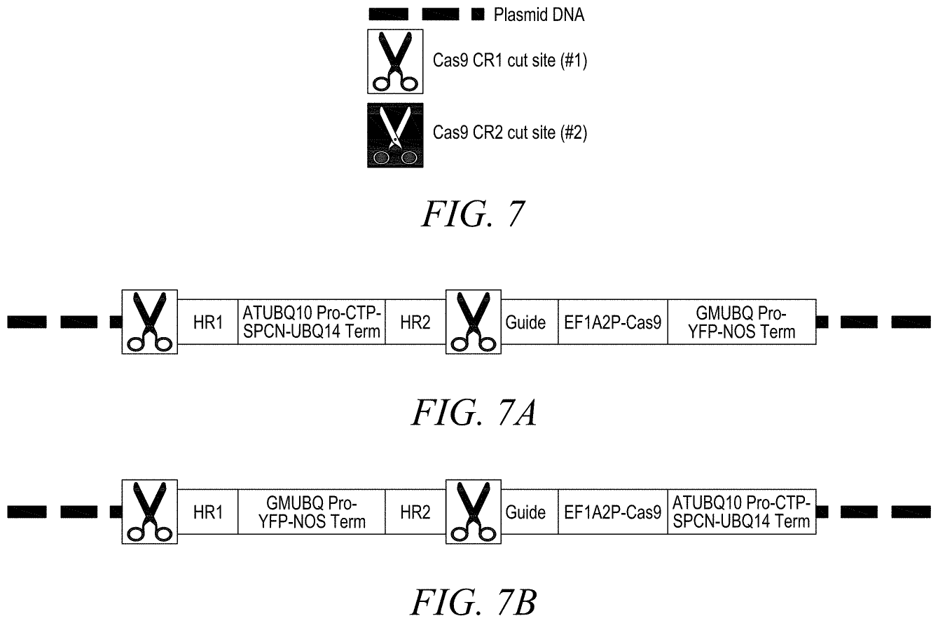

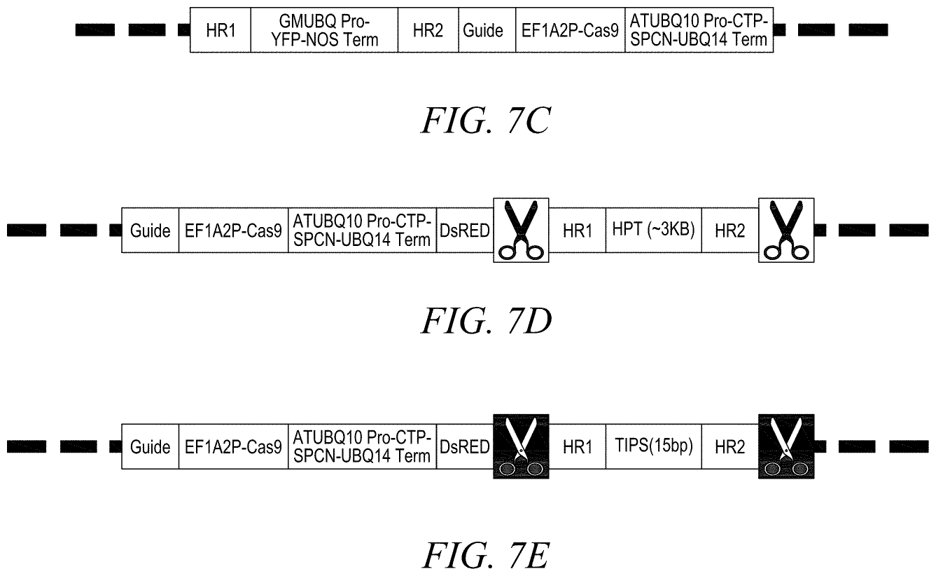

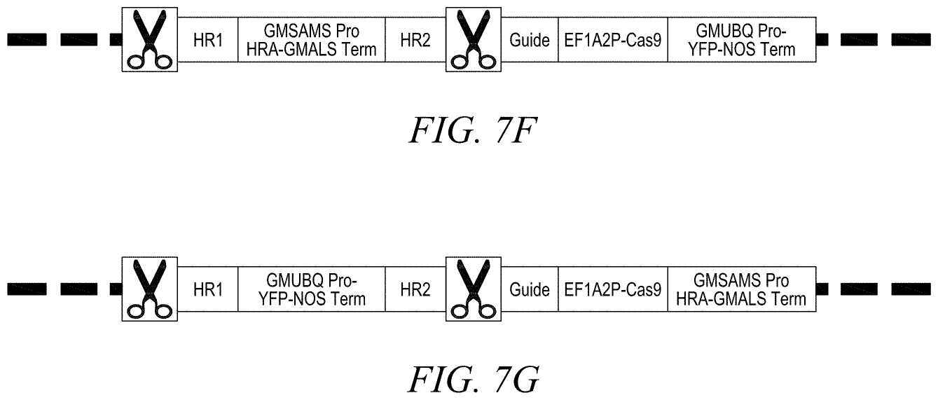

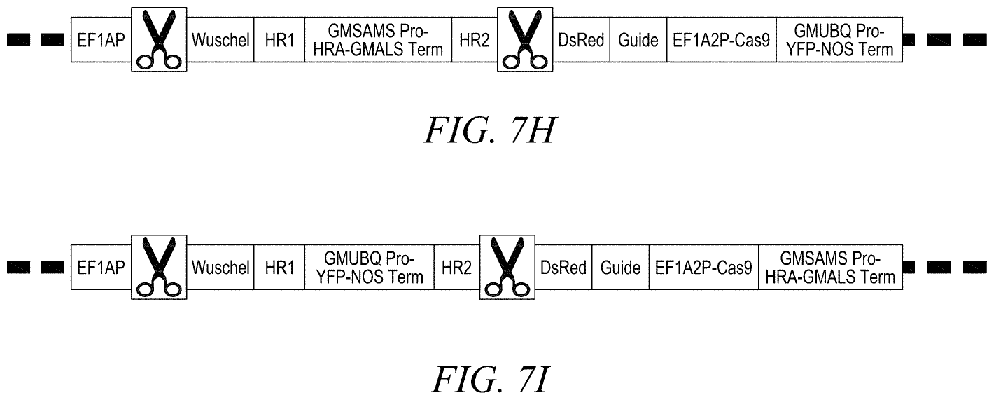

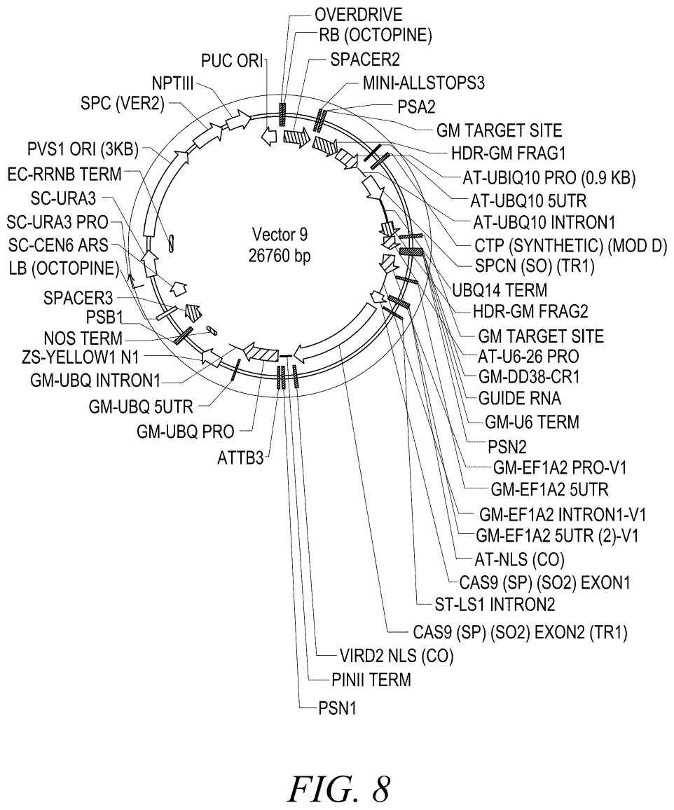

Methods of detecting the modified polynucleotides are provided. Methods of extracting modified DNA from a sample or detecting the presence of DNA corresponding to the modified genomic sequences comprising deletions of FAD2-1 and FAD3, such as presented in FIG. 3A-FIG. 3B, FIG. 4A-FIG. 4B, and FIG. 5A-FIG. 5B can be carried out. Such methods comprise contacting a sample comprising soybean genomic DNA with a DNA primer set, that when used in a nucleic acid amplification reaction, such as the polymerase chain reaction (PCR), with genomic DNA extracted from soybeans produces an amplicon that is diagnostic for either the presence or absence of the modified FAD2-1A and FAD3 alleles. The methods include the steps of performing a nucleic acid amplification reaction, thereby producing the amplicon and detecting the amplicon.

In some embodiments, one of the pair of DNA molecules comprises the wild type sequence where the modification occurs with the second of the pair being upstream or downstream as appropriate and suitably in proximity to the wild type sequence where the modification occurs, such that an amplicon is produced when the wild type allele is present, but no amplicon is produced when the modified allele is present. Suitable primers and probes for use in reactions to detect the presence of the alleles of FAD2-1A (e.g. SEQ ID NOs: 11, 12 and 13 and functional fragments thereof), FAD2-1B (e.g. SEQ ID NOs: 14, 12 and 15 and functional fragments thereof), FAD3a (e.g. SEQ ID NOs: 16, 17 and 18 and functional fragments thereof) and FAD3b (e.g. SEQ ID NOs: 19, 17 and 18 and functional fragments thereof) are provided in Table 3 and described in Example 4. In the context of the methods, in proximity means sufficiently close such that the distance between the first and second of the pair of DNA molecules facilitates the production of an amplicon when included in a DNA amplification reaction comprising soybean genomic DNA. For example, the second primer may bind at a location beginning at, within or less than 1, 2, 5, 10, 20, 30, 40, 50, 60, 70, 80, 90, 100, 150, 200, 250, 300, 350, 400, 450, 500, 600, 700, 800, 900, 1000, 1500, 16, 1700, 1800, 1900, 2000, 2100, 2200, 2300, 2400, 2500, 2600, 2700, 2800, 2900, 3000, 3500, 4000, 4500 or 5000 nucleotides upstream or downstream of the end of the binding site of the first DNA primer molecule.

Probes and primers are provided which are of sufficient nucleotide length to bind specifically to the target DNA sequence under the reaction or hybridization conditions. Suitable probes and primers are at least 10, 11, 12, 13, 14, 15, 16, 17, 18, 19, 20, 21, 22, 23, 24, 25, 26, 27, 28, 29 or 30 nucleotides in length, and less than 35, 34, 33, 32, 31, 30, 29, 28, 27, 26, 2, 5 2, 4 23, 22, 21, 20, 19, 18, 17, 16, 15, 14, 13, or 12 nucleotides in length. Such probes and primers can hybridize specifically to a target sequence under high stringency hybridization conditions. Preferably, probes and primers have complete or 100% DNA sequence similarity of contiguous nucleotides with the target sequence, although probes which differ from the target DNA sequence but retain the ability to hybridize to target DNA sequence may be also be used. Reverse complements of the primers and probes disclosed herein are also provided and can be used in the methods and compositions described herein.

In some embodiments, one of the pair of DNA molecules comprises the modification or traverses the modification junction such as the deletion junctions depicted in FIGS. 1-3, with the second DNA molecule of the pair being upstream or downstream of the genomic sequence as appropriate, such that an amplicon is produced when the modified allele is present, but no amplicon is produced when the wild type allele is present. Suitable primers for use in reactions to detect the presence of the modified alleles can be designed based on the junction sequences depicted in FIG. 3A—FIG. 3B, FIG. 4A—FIG. 4B, and FIG. 5A—FIG. 5B for the modified alleles.

For example, for SEQ ID NO: 55, the deletion junction occurs between positions 27 and 28; a primer can be designed which begins (or includes if beginning before position 1) at position 1 through position 27 and ends (or includes if ending after position 55) at position 29 to 55, provided that the primer is of sufficient length to function in the amplification reaction. The reverse complement of such a primer is also provided.

For example, for SEQ ID NO: 56, the deletion junction occurs between positions 23 and 24; a primer can be designed which begins (or includes if beginning before position 1) at position 1 through position 23 and ends (or includes if ending after position 55) at position 24 to 55, provided that the primer is of sufficient length to function in the amplification reaction. The reverse complement of such a primer is also provided.

For example, for SEQ ID NO: 47, the deletion junction occurs between positions 27 and 28; a primer can be designed which begins (or includes if beginning before position 1) at position 1 through position 27 and ends (or includes if ending after position 57) at position 28 to 57, provided that the primer is of sufficient length to function in the amplification reaction. The reverse complement of such a primer is also provided.

For example, for SEQ ID NO: 61, the deletion junction occurs between positions 27 and 28; a primer can be designed which begins (or includes if beginning before position 1) at position 1 through position 27 and ends (or includes if ending after position 55) at position 29 to 55, provided that the primer is of sufficient length to function in the amplification reaction. The reverse complement of such a primer is also provided.

For example, for SEQ ID NO: 70, the deletion junction occurs between positions 20 and 21; a primer can be designed which begins (or includes if beginning before position 1) at position 1 through position 20 and ends (or includes if ending after position 60) at position 22 to 60, provided that the primer is of sufficient length to function in the amplification reaction. The reverse complement of such a primer is also provided.

For example, for SEQ ID NO: 74, the deletion junction occurs between positions 20 and 21; a primer can be designed which begins (or includes if beginning before position 1) at position 1 through position 20 and ends (or includes if ending after position 58) at position 21 to 58, provided that the primer is of sufficient length to function in the amplification reaction. The reverse complement of such a primer is also provided.

For example, for SEQ ID NO: 77, the deletion junction occurs between positions 20 and 21; a primer can be designed which begins (or includes if beginning before position 1) at position 1 through position 20 and ends (or includes if ending after position 60) at position 22 to 60, provided that the primer is of sufficient length to function in the amplification reaction. The reverse complement of such a primer is also provided.

According to another aspect of the invention, methods of detecting the presence of a DNA molecule corresponding to the modified FAD2-1 and FAD3 alleles in a sample, include contacting the sample comprising DNA extracted from a soybean plant, cell or seed with a DNA probe molecule that hybridizes under stringent hybridization conditions with genomic DNA from a soybean comprising the modified FAD2-1 or FAD3 alleles and does not hybridize under stringent hybridization conditions with a control soybean plant DNA, The sample and probe are subjected to stringent hybridization conditions and hybridization of the probe to the DNA from the soybean plant, cell or seed comprising the modified FAD2-1 or FAD3

In some embodiments, the primers and probes bind and traverse the modification, such as a deletion junction, in the genomic DNA and have a sequence following the modification or junction in the 5′ to 3′ direction which is less than 10, 9, 8, 7, 6, 5, 4, 3, or 2 nucleotides in length and at least 1, 2, 3, 4 or 5 nucleotides in length.

### EXAMPLES

The present disclosure is further defined in the following Examples, in which parts and percentages are by weight and degrees are Celsius, unless otherwise stated. It should be understood that these Examples, while indicating embodiments of the invention, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Such modifications are also intended to fall within the scope of the appended claims.

### Example 1

### Soybean Optimized Expression Cassettes for Guide RNA/Cas Endonuclease Based Genome Modification in Soybean Plants

To use the guide RNA/Cas endonuclease system in soybean, the Cas9 gene from Streptococcus pyogenes M1 GAS (SF370) was soybean codon optimized per standard techniques known in the art. To facilitate nuclear localization of the Cas9 protein in soybean cells, Arabidopsis gene At3g04980 (amino acid 3118-3124) monopartite amino terminal nuclear localization signal AT-NLS (PKKKRKV, SEQ ID NO: 2) and Agrobacterium tumefaciens bipartite VirD2 T-DNA border endonuclease carboxyl terminal nuclear localization signal (KRPRDRHDGELGGRKRAR, SEQ ID NO: 3) were incorporated at the amino and carboxyl-termini of the Cas9 open reading frame, respectively. The soybean optimized Cas9 gene was operably linked to a soybean constitutive promoter such as the soybean constitutive promoter GM-EF1A2 disclosed in US Patent Publication No. 20090133159, incorporated herein by reference in its entirety (SEQ ID NO: 4) by standard molecular biological techniques.

The second component necessary to form a functional guide RNA/Cas endonuclease system for genome engineering applications is a duplex of the crRNA and tracrRNA molecules or a synthetic fusing of the crRNA and tracrRNA molecules, a guide RNA. To confer efficient guide RNA expression (or expression of the duplexed crRNA and tracrRNA) in soybean, the soybean U6 polymerase III promoter and U6 polymerase III terminator were used.

Approximately 0.5 kb genomic DNA sequence upstream of the first G nucleotide of a U6 gene was selected to be used as a RNA polymerase III promoter for example, GM-U6-13.1 promoter (SEQ ID NO:5) or GM-U6-9.1 promoter (SEQ ID NO:6), to express guide RNA to direct Cas9 nuclease to designated genomic site. The guide RNA coding sequence was 76 bp long and comprised a 20 bp variable targeting domain from a chosen soybean genomic target site on the 5′ end and a tract of 4 or more T residues as a transcription terminator on the 3′ end. The first nucleotide of the 20 bp variable targeting domain was a G residue to be used by RNA polymerase III for transcription. Other soybean U6 homologous genes promoters were similarly cloned and used for small RNA expression.

Since the Cas9 endonuclease and the guide RNA need to form a protein/RNA complex to mediate site-specific DNA double strand cleavage, the Cas9 endonuclease and guide RNA must be expressed in same cells. To improve their co-expression and presence, the Cas9 endonuclease and guide RNA expression cassettes were linked into a single DNA construct.

### Example 2

### Selection of Soybean FAD2 and FAD3 Target Sites to be Cleaved by the Guide RNA/Cas Endonuclease System

A. guideRNA/Cas9 Endonuclease Target Site Design on the Soybean FAD2-1 and FAD3 Genes.

There are two seed preferred FAD2-1 genes in soybean (FAD2-1A for Glyma.10g278000 and FAD2-1B for Glyma.20g111000). One guide RNA/Cas9 endonuclease target site (GM-FAD2-1 CR1) was designed to target both the FAD2-1 genes (Table 2). There are also two major FAD3 genes in soybean (FAD3a for Glyma.14g194300 and FAD3b for Glyma.02g227200). The GM-FAD3 CR2 site was designed to target both FAD3 genes (Table 2).

TABLE 2
Guide RNA/Cas9 endonuclease target sites
on soybean FAD2-1 genes and FAD3 genes

| Name of gRNA- | Cas endonuclease | |
|---|---|---|
| Cas9 endonuclease | target sequence | |
| target site | (SEQ ID NO:) | Physical location |
| GM-FAD2-1 CR1 | 7 | Gm10:50014185 . . . 50014166 |
| | | Gm20:35317773 . . . 35317754 |
| GM-FAD3 CR2 | 8 | Gm14:45939600 . . . 445939618 |
| | | Gm02:41423563 . . . 41423581 |

### B. Guide-RNA Expression Cassettes, Cas9 Endonuclease Expression Cassettes and Knockout of the Soybean FAD2-1 and FADS Genes.

The soybean U6 small nuclear RNA promoter, GM-U6-13.1 (SEQ ID NO: 5) or GM-U6-9.1 promoter (SEQ ID NO:6), was used to express guide RNAs to direct Cas9 nuclease to designated genomic target sites. A soybean codon optimized Cas9 endonuclease with a potato ST-LS1 Intron2 (SEQ ID NO: 1) expression cassette and one or two guide RNA expression cassettes were linked in the binary plasmids. In total, 4 binary vectors were made (FIG. 1A—FIG. 1D). In the RV019927 construct (SEQ ID NO.9) and RV019928 construct (SEQ ID NO.31), the GM-FAD2-1 CR1 gRNA expression cassette and the Cas9 expression cassette was made to target both the FAD2-1A and FAD2-1B genes simultaneously. Similarly, the RV019929 construct (SEQ ID NO.10) and RV019930 construct (SEQ ID NO.32) were made to target the FAD2-1A, FAD2-1B, FAD3a and Fad3b genes at the same time. The binary vectors, such as the RV019927 or RV019928, or RV019929, or RV019930 were transformed into Ochrobactrum haywardense H1-8 strain that has a helper plasmid RV005393 (SEQ ID NO.30) for soybean transformation.

### Example 3

### Delivery of the Guide RNA/Cas9 Endonuclease System DNA to Soybean by Ochro-EA Stable Transformation

Ochrobactrum-mediated soybean embryonic axis transformation was done essentially as described in US Patent Publication No. 2018/0216123, incorporated herein by reference in its entirety. Mature dry seeds of soybean cultivar 93Y21 were disinfected using chlorine gas and imbibed on semi-solid medium containing 5 g/l sucrose and 6 g/l agar at room temperature in the dark. After an overnight incubation, the seed was soaked in distilled water for an additional 3-4 hrs at room temperature in the dark. Intact embryonic axis were isolated from cotyledon using a scapel blade in distilled sterile water. The embryonic axis explants were transferred to the deep plate with 15 mL of Ochrobactrum haywardense H1-8 containing a helper vector PHP85634 (RV005393) with binary vector RV019927 or RV019928, or RV019929, or RV019930 with suspension at OD600=0.5 in infection medium containing 200 μM acetosyringone. The plates were sealed with parafilm (“Parafilm M” VWR Cat #52858), then sonicated (Sonicator-VWR model 50T) for 30 seconds. After sonication, embryonic axis explants were transferred to a single layer of autoclaved sterile filter paper (VWR #415/Catalog #28320-020). The plates were sealed with Micropore tape (Catalog #1530-0, 3M, St. Paul, Minn.)) and incubated under dim light (5-10 μE/m²/s, cool white fluorescent lamps) for 16 hrs at 21° C. for 3 days.

After co-cultivation, the embryonic axis explants were cultured on shoot induction medium solidified with 0.7% agar in the absence of selection. The base of the explant (i.e., root radical of embryonic axis) was embedded in the medium. Shoot induction was carried out in a Percival Biological Incubator at 26° C. with a photoperiod of 18 hrs and a light intensity of 40-70 μE/m²/s. 6 to 7 weeks after transformation, elongated shoots (>1-2 cm) were isolated and transferred to rooting medium containing a selection agent. Transgenic plantlets were transferred to soil pots and were grown in the greenhouse.

### Example 4

### Detection of Site-Specific Mutations with NHEJ Mediated by the Guide RNA/Cas9 System in Stably Transformed Soybean

Genomic DNA was extracted from somatic embryo samples and analyzed by quantitative PCR using a 7500 real time PCR system (Applied Biosystems, Foster City, Calif.) with target site-specific primers and a FAM-labeled fluorescence probe to check copy number changes of the target sites. The qPCR analysis was done in duplex reactions with a syringolide induced protein (SIP) gene as the endogenous control and a wild type 93Y21 genomic DNA sample that contains one copy of the target site with 2 alleles, as the single copy calibrator. The endogenous control probe SIP-T was labeled with VIC (2′-chloro-7′phenyl-1,4-dichloro-6-carboxy-fluorescein) and the gene-specific probes FAD2-T1, FAD2-T2 and FAD3-T2 were labeled with FAM (Fluorescein) (Table 3) for the simultaneous detection of both fluorescent probes (Applied Biosystems). PCR reaction data were captured and analyzed using the sequence detection software provided with the 7500 real time PCR system and the gene copy numbers were calculated using the relative quantification methodology (Applied Biosystems).

TABLE 3
Primers/probes used in qPCR analyses of
transgenic soybean events.

| Target | Primer/ | | SEQ ID |
|---|---|---|---|
| Site | Probe Name | Sequences | NOs: |
| FAD2-1A | FAD2-F1 | TCGTGTGGCCAAAGTGGAA | 11 |
| | FAD2-R1 | TTTGTGTTTGGAACCCTTGAGA | 12 |
| | FAD2-T1 | TTCAAGGGAAGAAGCC | 13 |
| | (FAM-MGB) | | |
| FAD2-1B | FAD2-F2 | CCGTGTGGCCAAAGTTGAA | 14 |
| | FAD2-R1 | TTTGTGTTTGGAACCCTTGAGA | 12 |
| | FAD2-T2 | TTCAGCAGAAGAAGCC | 15 |
| | (FAM-MGB) | | |
| FAD3a | FAD3-F1 | TAATGGATACCAAAAGGAAGC | 16 |
| | FAD3-R2 | CAAGCACATCCCTGAGAACATAA | 17 |
| | | C | |
| | FAD3-T2 | AATCCATGGAGATCCCT | 18 |
| | (FAM-MGB | | |
| FAD3b | FAD3-F2 | ATACCAACAAAAGGGTTCTTC | 19 |
| | FAD3-R2 | CAAGCACATCCCTGAGAACATAA | 17 |
| | | C | |
| | FAD3-T2 | AATCCATGGAGATCCCT | 18 |
| | (FAM-MGB) | | |
| SIP | SIP-130F | TTCAAGTTGGGCTTTTTCAGAAG | 20 |
| | SIP-198R | TCTCCTTGGTGCTCTCATCACA | 21 |
| | SIP-170T | CTGCAGCAGAACCAA | 22 |
| | (VIC-MGB) | | |

Since the wild type 93Y21 genomic DNA with two alleles of the target site was used as the single copy calibrator, events without any change of the target site would be detected as one copy herein termed Wt-Homo (qPCR value >=0.7), events with one allele changed, which is no longer detectible by the target site-specific qPCR, would be detected as half copy herein termed NHEJ-Hemi (qPCR value between 0.1 and 0.7), while events with both alleles changed would be detected as null herein termed NHEJ-Null (qPCR value=<0.1).

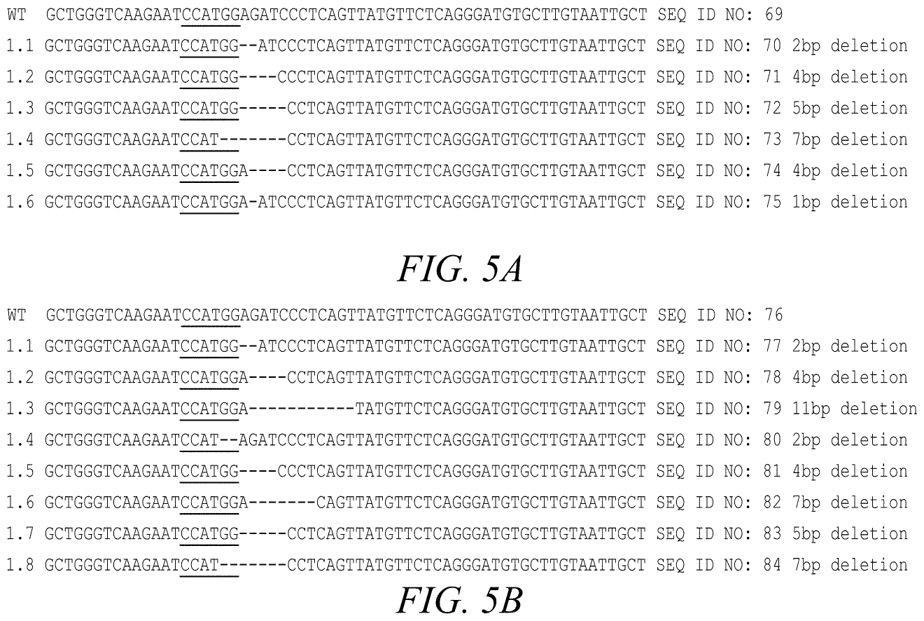

In total, four soybean transformation experiments were carried out with the vectors shown FIG. 1A-FIG. 1D. In RV019927 and RV019929, the gRNA cassettes were near the right border and placed upstream of the Cas9 expression cassettes in the binary vectors. In contrast, in RV019928 and RV019930, the gRNA expression cassettes were near the left border and placed downstream of the Cas9 expression cassettes in the binary vectors. As shown in Table 4, Table 5 and FIG. 2, for the two experiments to knockout only the two FAD2-1 genes, the gRNA near the right border and placed upstream of Cas9 configuration design provided much higher gene knockout efficiency as compared to the design with the gRNA near the left border and placed downstream of Cas9. For example, the bi-allelic (NHEJ-Null) knockout efficiency reached 63% for the FAD2-1A gene and 56% for the FAD2-1B gene (Table 4) with the RV019927 vector. The bi-allelic (NHEJ-Null) knockout efficiency was only 7% for the FAD2-1 gene and 10% for the FAD2-1B gene (Table 5) with the RV019928 vector. The WT population only made up about 2-3% in the experiment with the RV019927 vector, as compared to 37-44% in the experiment with the RV019928 vector.

In the third and fourth experiments, either the RV019929 or RV019930 binary vectors was used to knockout the two FAD2-1 genes and the two FAD3 genes. As shown in Table 4, Table 5 and FIG. 2, these two experiments also demonstrated that RV019929 vector design, with the gRNA expression cassette near the right border and upstream of the Cas9 expression cassette, provided much higher quadra gene knockout efficiency as compared to the RV019930 binary vector in the FAD2-1A, FAD2-1B, FAD3a and FAD3b genes. These unexpected results demonstrated the different gRNA/Cas9 expression cassette configurations in the binary vectors had dramatic effects on the target gene editing efficiency. Vectors with gRNA cassettes near the right border and placed upstream of the Cas9 expression cassettes increased the efficiency of editing the target site.

TABLE 4
FAD2-1 Target Site Mutations Induced by the Guide RNA/Cas9
system in 93Y21 with RV019927. Numbers indicate no.
of events (numbers in parentheses are %).

| | Total | Wt-Homo | NHEJ-Hemi | NHEJ-Null |
|---|---|---|---|---|
| Target site | events | (%) | (%) | (%) |
| FAD2-1A | 106 | 2 (2%) | 37 (35%) | 67 (63%) |
| FAD2-1B | | 3 (3%) | 44 (42%) | 59 (56%) |

TABLE 5
FAD2-1 Target Site Mutations Induced by the Guide RNA/Cas9
system in 93Y21 with RV019928. Numbers indicate no.
of events (numbers in parentheses are %).

| | Total | Wt-Homo | NHEJ-Hemi | NHEJ-Null |
|---|---|---|---|---|
| Target site | event | (%) | (%) | (%) |
| FAD2-1A | 41 | 15 (37%) | 23 (56%) | 3 (7%) |
| FAD2-1B | | 18 (44%) | 19 (46%) | 4 (10%) |

TABLE 6
FAD2-1 and FAD3 Target Site Mutations Induced by
the Guide RNA/Cas9 system in 93Y21 with RV019929.
Numbers indicate no. of events (numbers in parentheses
are % of the total analyzed events).

| | Total | Wt-Homo | NHEJ-Hemi | NHEJ-Null |
|---|---|---|---|---|
| Target Site | event | (%) | (%) | (%) |
| FAD2-1A | 27 | 0 (0%) | 11 (41%) | 16 (59%) |
| FAD2-1B | | 1 (4%) | 12 (44%) | 14 (52%) |
| FAD3a | | 1 (4%) | 6 (22%) | 20 (74%) |
| FAD3b | | 1 (4%) | 4 (15%) | 22 (81%) |

TABLE 7
FAD2-1 and FAD3 Target Site Mutations Induced by
the Guide RNA/Cas9 system in 93Y21 with RV019930.
Numbers indicate no. of events (numbers in parentheses
are % of the total analyzed events).

| | Total | Wt-Homo | NHEJ-Hemi | NHEJ-Null |
|---|---|---|---|---|
| Target Site | event | (%) | (%) | (%) |
| FAD2-1A | 42 | 20 (48%) | 17 (40%) | 5 (12%) |
| FAD2-1B | | 7 (17%) | 32 (76%) | 3 (7%) |
| FAD3a | | 7 (17%) | 32 (76%) | 3 (7%) |
| FAD3b | | 12 (29%) | 22 (52%) | 8 (19%) |

The target regions of NHEJ-Null events were amplified by regular PCR from the same genomic DNA samples using primers specific respectively to FAD2-1A, FAD2-1B, FAD3a and FAD3b genes (Table 8).

TABLE 8
PCR primers for the gRNA targets of the FAD2-1 and FAD3 genes

| Target Site | Primer1 | SEQ ID NO: | Primer2 | SEQ ID NO: |
|---|---|---|---|---|
| FAD2-1A | WOL1007 | 23 | WOL1009 | 25 |
| FAD2-1B | WOL1008 | 24 | WOL1009 | 25 |
| FAD3a | WOL1100 | 26 | WOL1101 | 27 |
| FAD3b | WOL1102 | 28 | WOL1103 | 29 |

The PCR bands were cloned into pCR2.1 vector using a TOPO-TA cloning kit (Invitrogen) and multiple clones were sequenced to check for target site sequence changes as the results of NHEJ. Various small deletions near the Cas9 cleavage site, 3 bp upstream of the PAM, were revealed at all four tested target sites, with most of them resulting in frame-shift knockouts (FIG. 3A-FIG. 3B, FIG. 4A-FIG. 4B, FIG. 5A-FIG. 5B). These sequence analyses confirmed the occurrence of NHEJ mediated by the guide RNA/Cas9 system at the specific Cas9 target sites.

### Example 5

### Soybean Vectors for SDN2 and SDN3

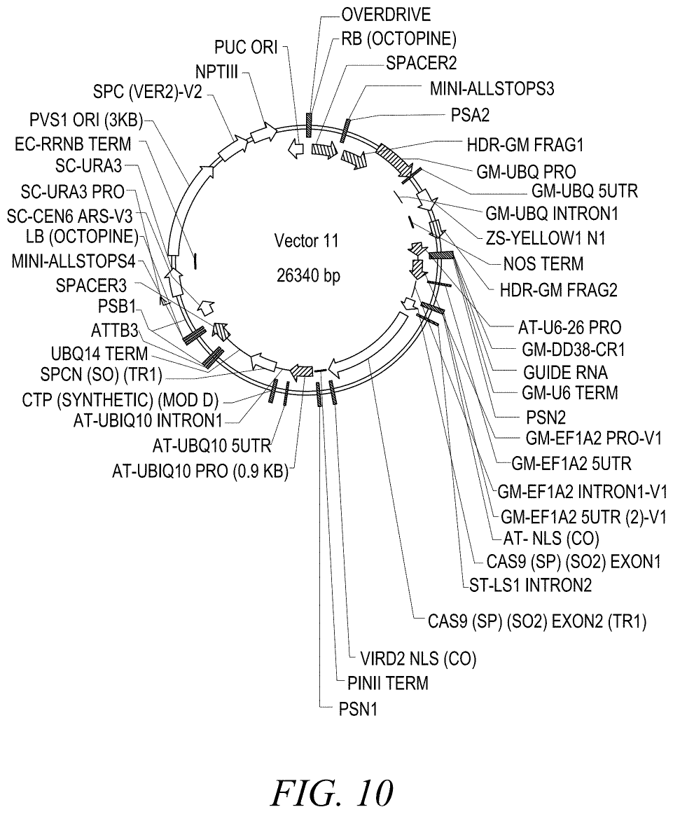

The T-DNA vectors used in the Cas9 soybean experiments were Vectors 9-17, depicted in FIGS. 7A-7I and FIGS. 8-16, and given as SEQ ID NOs: 97-105, respectively. The lengths of the homology region “arms” (HR1 and HR2) in each vector ranged from 591 to 980 nucleotides in length.

### Example 6

### Soy Transformation

Exemplary protocols for Agrobacterium-mediated transformation disclosed in Jia et al., 2015, Int J. Mol. Sci. 16:18552-18543 and US20170121722 and incorporated herein by reference in their entireties), or Ochrobactrum-mediated transformation, disclosed in US20180216123A1 and incorporated herein by reference in its entirety, for soybean can be used with the methods of the disclosure.

Soybean transformation was done essentially as described in US20180216123A1 and in US20170121722 and incorporated herein by reference in their entireties or as described by Paz et al. ((2006) Plant Cell Rep 25:206-213) and U.S. Pat. No. 7,473,822 and incorporated herein by reference in its entirety. Mature seed from soybean lines were surface-sterilized for 16 hrs using chlorine gas, produced by mixing 3.5 mL of 12 N HCl with 100 mL of commercial bleach (5.25% sodium hypochloride), as described by Di et al. ((1996) Plant Cell Rep 15:746-750). Disinfected seeds were soaked in sterile distilled water at room temperature for 16 hrs (100 seeds in a 25×100 mm petri dish).

A volume of 10 mL of Ochrobactrum haywardense H1 NRRL Deposit B-67078, disclosed in US20180216123A1 and incorporated herein by reference in its entirety, further containing vector PHP70365 (SEQ ID NO: 108) suspension at OD600=0.5 in infection medium containing 300 μM acetosyringone was added to the soaked seeds. The seeds were then split by cutting longitudinally along the hilum to separate the cotyledons, and the seed coats, primary shoots, and embryonic axes were removed from the Ochrobactrum haywardense H1 NRRL Deposit B-67078 suspension, thereby generating half-seed explants.

The half-seed explants were placed flat side down in a deep plate with 4 mL fresh Ochrobactrum/infection media with no overlapping of cotyledons. The plates were sealed with parafilm (“Parafilm M” VWR Cat #52858), then sonicated (Sonicator-VWR model 50T) for 30 seconds. After sonication, half-seed explants were transferred to a single layer of autoclaved sterile filter paper (VWR #415/Catalog #28320-020) onto co-cultivation solid medium (18-22 explants per plate; flat side down). The plates were sealed with Micropore tape (Catalog #1530-0, 3M, St. Paul, Minn.)) and incubated under dim light (5-10 μE/m²/s, cool white fluorescent lamps) for 16 hrs at 21° C. for 5 days.

### Example 7

### Soy Regeneration

Methods were carried out according to those disclosed in US20180216123A1, incorporated herein by reference in its entirety. After co-cultivation, the half-seed explants were washed in liquid shoot induction (SI) medium once then the explants were cultured on shoot induction medium solidified with 0.7% agar in the absence of selection (Table 10 of US20180216123A1, incorporated herein by reference in its entirety). The base of the explant (i.e., the part of the explant from where the embryonic axis was removed) was embedded in the medium, facing upwards. Shoot induction was carried out in a Percival Biological Incubator at 24° C. with a photoperiod of 18 hrs and a light intensity of 130-160 μE/m²/s. After 14 days, the explants were transferred to fresh shoot induction medium containing 3 mg/L bialaphos (Table 10 of US20180216123A1, incorporated herein by reference in its entirety). The half seed explants were transferred to fresh medium every two weeks. After four weeks of culture on shoot induction medium, explants were transferred to shoot elongation (SE) medium containing 5 mg/L bialaphos (Table 10 of US20180216123A1, incorporated herein by reference in its entirety). Six to ten weeks later, elongated shoots (>1-2 cm) were isolated and transferred to rooting medium (Table 10 of US20180216123A1, incorporated herein by reference in its entirety) containing 1 mg/L bialaphos.

### Example 8

### Analysis of HDR Frequency

### Soybean SDN3

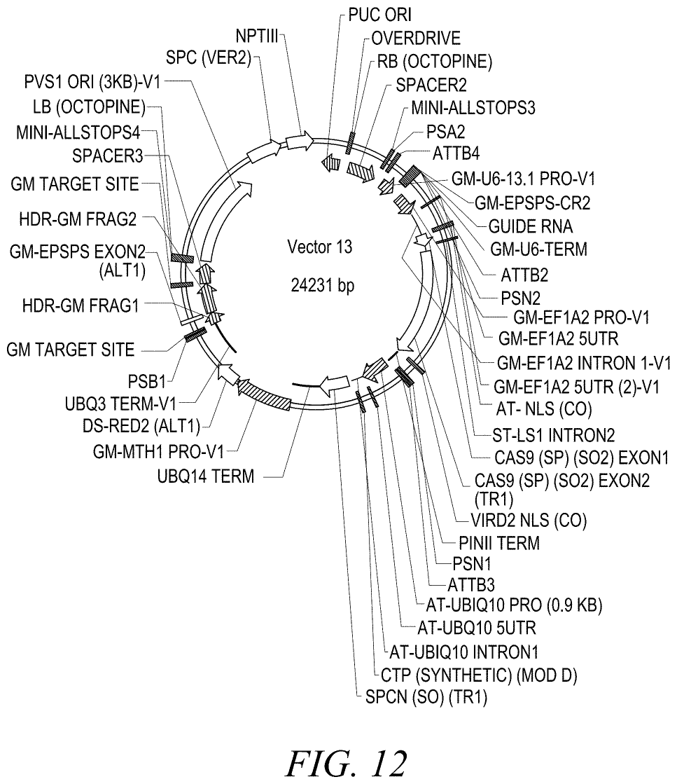

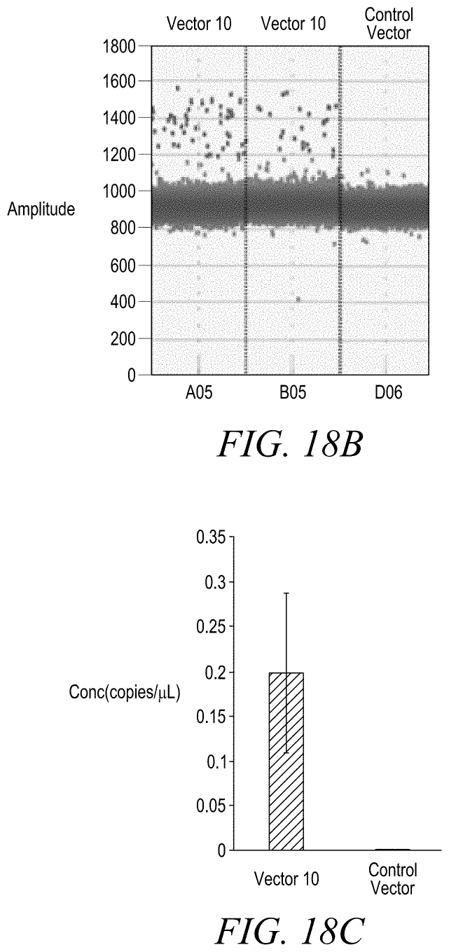

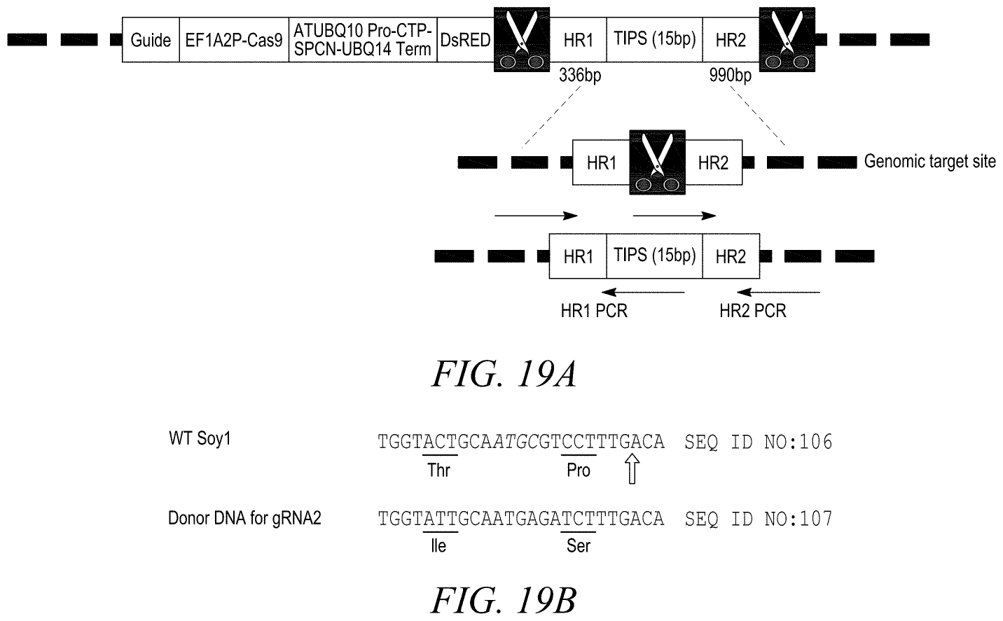

Ochrobactrum and Agrobacterium-mediated transformation and plant regeneration were performed as described in Examples 6 and 7. Several rapid testing experiments were conducted to evaluate feasibility of these improved SDN3 methods in soybean. Soybean embryonic axis infected with Ochrobactrumcontaining SDN3 donor in transformation vectors, Vector 9 (FIG. 7A, FIG. 8, and FIG. 17A) and Vector 10 (FIG. 7B, FIG. 9, and FIG. 18A), were sampled after 7 days for DNA extraction. Digital droplet PCR (ddPCR) for the HR2 junction revealed a positive signal for HDR using Vector 9 and Vector 10 while no HDR signal was detected from the control vector (FIGS. 17B-C and 18B-C, respectively).

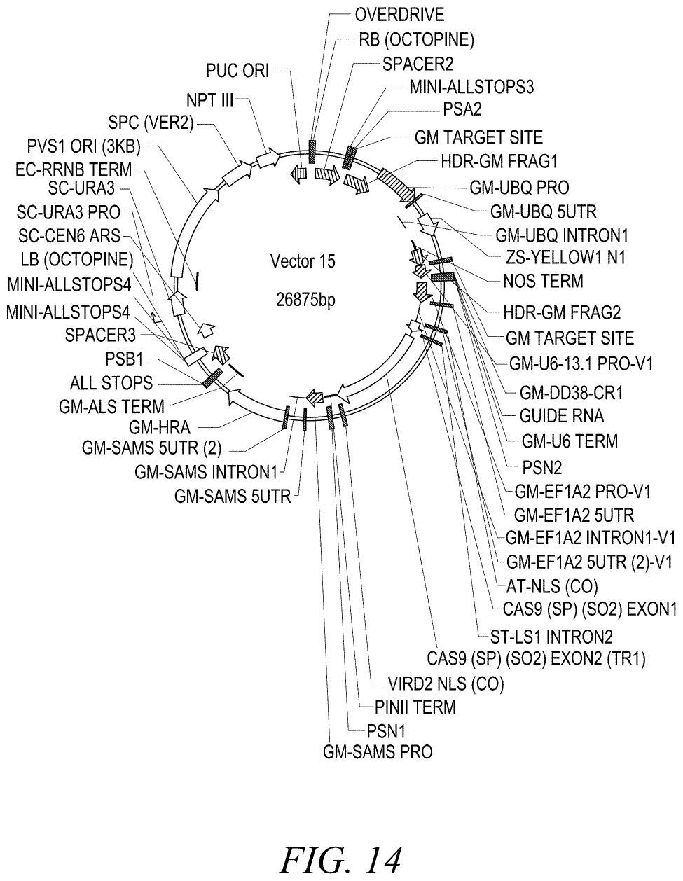

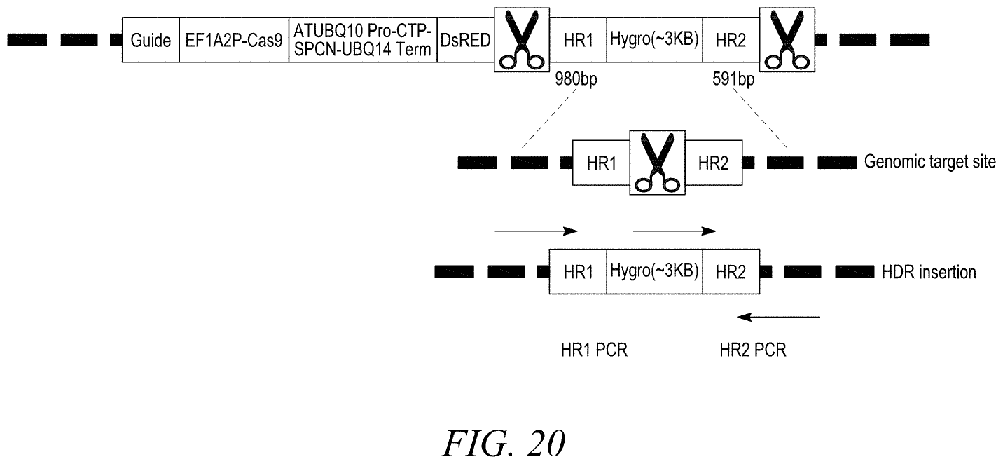

T0 soybean plants transgenic to vector Vector 12 (FIG. 11 and FIG. 20) were regenerated using the Spectinomycin (SPCN) gene as a selectable marker and analyzed by junction qPCR for targeted SDN3 insertion. 2×HDR positive events were further analyzed by sequencing to evaluate the size and integrity of the insertion. Table 9 shows the results.

TABLE 9
Frequencies of HDR-facilitated targeted SDN3 polynucleotide
modification edits at a target site in soy. Frequencies
are calculated based on the total number of plants
analyzed in each experiment.

| | Plants analyzed | 2X HDR | Frequency |
|---|---|---|---|
| Batch 1 | 222 | 7 | 3.2% |
| Batch 2 | 244 | 5 | 2% |
| Total | 466 | 12 | 2.6% |

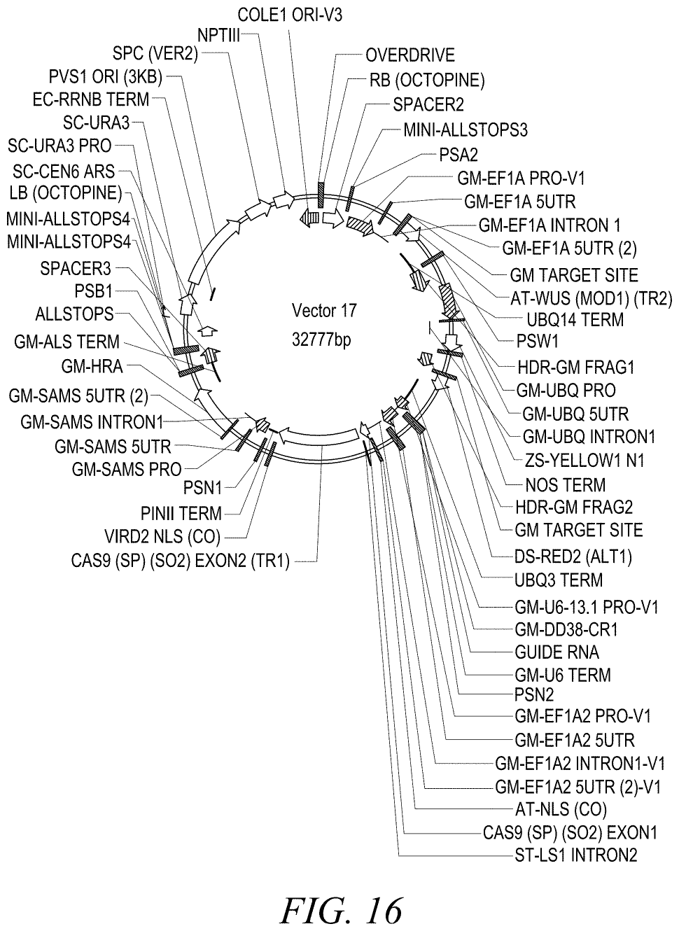

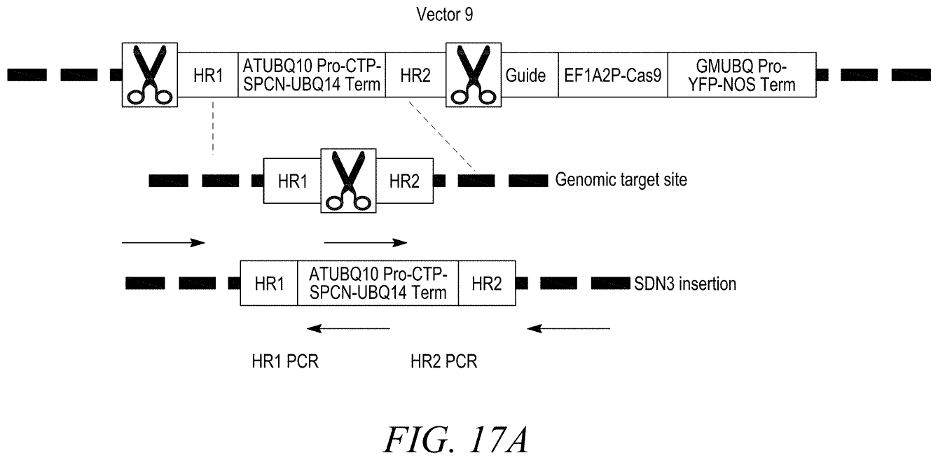

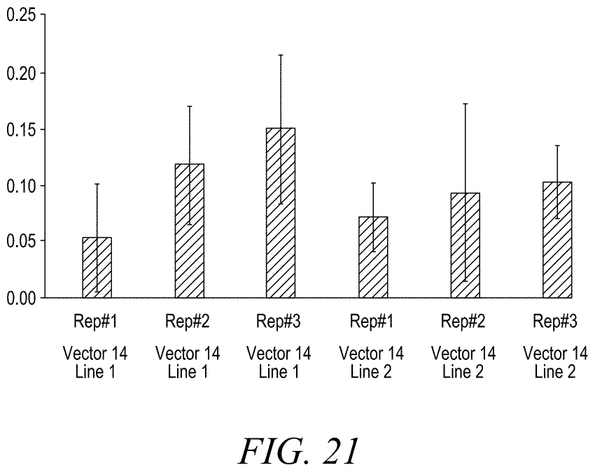

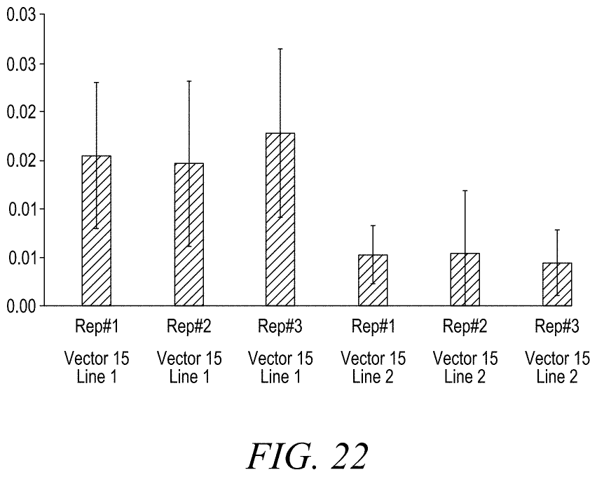

Soybean leaf explants from seedlings of two different genotypes were infected and co-cultivated for 3 days with Agrobacterium containing SDN3 donor in transformation vectors, Vector 14 (FIG. 7F, FIG. 13) and Vector 15 (FIG. 7G, FIG. 14). Leaf samples were sampled after 7 days for DNA extraction. Digital droplet PCR (ddPCR) for the HR2 junction revealed positive signal for HDR from both vectors (FIG. 21 for Vector 14; FIG. 22 for Vector 15).

### Soybean SDN2

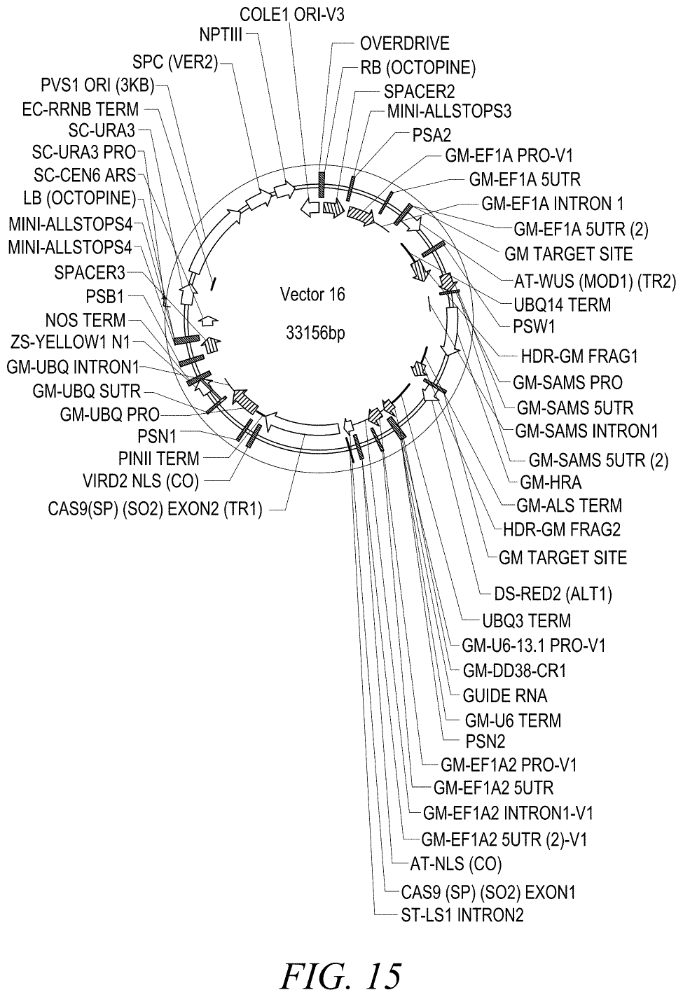

TO soybean plants transgenic to Vector 13 (FIG. 7E, FIG. 12, and FIG. 19A) were regenerated using the Spectinomycin (SPCN) gene as a selectable marker and analyzed by junction qPCR for targeted SDN2 insertion. 2×HDR positive events were further analyzed by sequencing to evaluate the size and integrity of the insertion. Of 1358 plants analyzed, 8 demonstrated editing, for a frequency of 0.6%. FIGS. 19C and 19D show sequence verification of the edits. Results are shown in Table 10.

TABLE 10
Frequencies of HDR-facilitated targeted SDN2 polynucleotide
modification edits at a target site in soy. Frequencies
are calculated based on the total number of plants
analyzed in each experiment.

| Plants analyzed | Edited | Frequency |
|---|---|---|
| 1358 | 8 | 0.6% |

These examples demonstrated robust Ochrobactrum- and Agrobacterium-mediated SDN2 and SDN3 systems in soybean with donor DNA cassette flanking with target sites resulting in releasing the donor DNA molecule from the T-DNA. The technological advances in SDN2 and SDN3 systems were made without using a selectable marker gene inside the donor template. Other types of genome modifications, such as targeted nucleotide editing and gene replacement (swap), will also benefit from this approach.

### Example 9

### Additional Methods

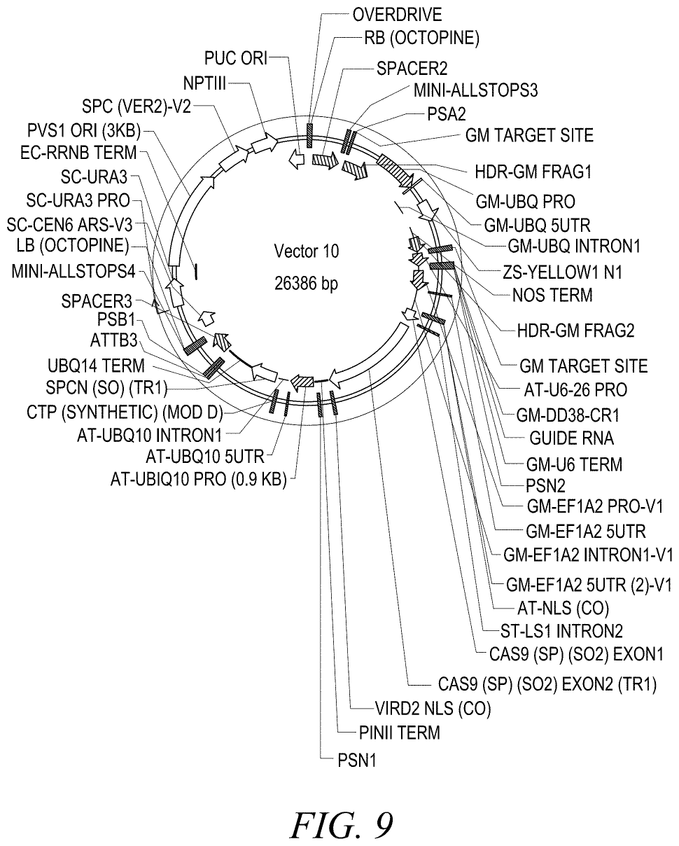

Efficient release of the donor DNA polynucleotide can be promoted by several methods. In one method, a plurality (n) of sets of sequences can be incorporated flanking the donor DNA cassette (one depiction for n=2 is depicted in FIG. 6), which allows multiple opportunities for cleavage from a Cas endonuclease/guide RNA complex. In some aspects, two sets of sequences flank the donor DNA cassette. In some aspects, three sets of sequences flank the donor DNA cassette. In some aspects, four or more sets of sequences flank the donor DNA cassette. The number of sets for the plurality may be n=2, 3, 4, 5, 6, 7, 8, 9, 10, or greater than 10.

Methods to improve the frequency of HDR at a target site may also include a donor/template cassette that has one target site outside of the cassette, instead of two sites flanking the cassette. This results in a “hanging” template/donor fragment provided to the target polynucleotide at or near the double-strand break site.

The foregoing disclosure has been described in detail by way of illustration and example for purposes of clarity and understanding. As is readily apparent to one skilled in the art, the foregoing are only some of the methods and compositions that illustrate the embodiments of the foregoing disclosure. It will be apparent to those of ordinary skill in the art that variations, changes, modifications, and alterations may be applied to the compositions and/or methods described herein without departing from the true spirit, concept, and scope of the disclosure.

All publications, patents, and patent applications mentioned in the specification are incorporated by reference herein for the purpose cited to the same extent as if each was specifically and individually indicated to be incorporated by reference herein.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth. Unless expressly stated to the contrary, “or” is used as an inclusive term. For example, a condition A or B is satisfied by any one of the following: A is true (or present) and B is false (or not present), A is false (or not present) and B is true (or present), and both A and B are true (or present).

1. A method for increasing the efficiency of editing a target site in a plant, the method comprising:
(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary to the target site, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage at the target site;
(b) identifying at least one plant cell of (a) that has a modification at the target site, wherein the modification includes at least one deletion or substitution of one or more nucleotides at the target site; and
(c) regenerating a plant from the at least one plant cell of (b) having the modification at the target site having increased editing efficiency when compared to a control plant having a modification at the target site provided by a control plant editing T-DNA, wherein the control plant editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary to the target site and wherein the guide RNA and the Cas endonuclease are capable of forming a complex that enables the Cas endonuclease to introduce cleavage at the target site.

2. A method of increasing the efficiency of altering the fatty acid profile in the seed of a plant, the method comprising:
(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage in the FAD2 genomic sequence in the plant genome, the FAD3 genomic sequence in the plant genome, or a combination thereof;
(b) obtaining a plant from the plant cell of (a);
(c) evaluating the plant of (b) for the presence of the at least one nucleotide modification;
(d) selecting a progeny plant of (c) having an altered fatty acid profile; and
(e) obtaining seed from the progeny plant of (d) having increased editing efficiency when compared to a control seed of a plant having a modification of the FAD2 genomic sequence, the FAD3 genomic sequence, or the combination thereof provided by a control seed editing T-DNA, wherein the control seed editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof wherein the guide RNA and the Cas endonuclease are capable of forming a complex that enables the Cas endonuclease to introduce cleavage in the FAD2 genomic sequence in the plant genome, the FAD3 genomic sequence in the plant genome, or a combination thereof.

3. A method of increasing the efficiency of altering the fatty acid profile in the seed of a plant, the method comprising:
(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage within the FAD2 genomic sequence in the plant genome, the FAD3 genomic sequence in the plant genome, or a combination thereof;
(b) obtaining a plant from the plant cell of (a);
(c) evaluating the plant of (b) for the presence of the at least one nucleotide modification;
(d) screening a progeny plant of (c) having an altered fatty acid profile that is void of the guide RNA and the Cas endonuclease; and
(e) obtaining seed from the progeny plant of (d) having increased editing efficiency when compared to a control seed of a plant having a modification of the FAD2 genomic sequence, the FAD3 genomic sequence, or the combination thereof provided by a control seed editing T-DNA, wherein the control seed editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof, and wherein the guide RNA and the Cas endonuclease are capable of forming a complex that enables the Cas endonuclease to introduce cleavage within the FAD2 genomic sequence in the plant genome, the FAD3 genomic sequence in the plant genome, or a combination thereof.

4. A method for increasing the efficiency of introducing a nucleotide of interest into a target site in the genome of a plant, the method comprising:
(a) providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to cleavage at the target site;
(b) contacting the plant cell of (a) with a donor DNA comprising the polynucleotide of interest;
(c) identifying at least one plant cell from (b) comprising in its genome the polynucleotide of interest integrated at the target site; and
(d) regenerating a plant from the at least one plant cell having in its genome the polynucleotide of interest integrated at the target site having increased efficiency of introduction of the polynucleotide of interest into the target site in the genome of the plant cell when compared to a control plant having an introduction of the polynucleotide of interest into the target site in the genome of the plant cell provided by a control plant editing T-DNA, wherein the control plant editing T-DNA comprises in operable linkage from a right border to a left border orientation, a Cas endonuclease expression cassette, wherein the Cas endonuclease expression cassette comprises a regulatory element operably linked to a Cas endonuclease and a guide RNA expression cassette wherein, the guide RNA expression cassette comprises a regulatory element operably linked to a polynucleotide encoding a guide RNA, wherein the guide RNA and the Cas endonuclease are capable of forming a complex that enables the Cas endonuclease to cleavage at the target site.

5. The method of claim 1, the method further comprising a trait of interest or a nucleotide modification template cassette, wherein the trait of interest or nucleotide modification template cassette comprises at least one nucleotide modification of the trait of interest or nucleotide and the trait of interest or nucleotide modification template cassette is capable of making at the least one nucleotide modification at the target site of the trait of interest or the nucleotide.

6. The method of claim 2, the method further comprising a modification template cassette, wherein the modification template cassette comprises at least one nucleotide modification of a FAD2 genomic sequence in the plant genome, a FAD3 genomic sequence in the plant genome, or a combination thereof and the modification template cassette enables the at least one nucleotide modification of the FAD2 genomic sequence in the plant genome, the FAD3 genomic sequence in the plant genome, or the combination thereof.

7. The method of claim 1, wherein the target site is selected from the group consisting of a promoter sequence, a terminator sequence, a regulatory element sequence, a coding sequence, a splice site, a polyubiquitination site, an intron site, an intron enhancing motif, a gene of interest, and a trait of interest.

8. The method of claim 1, wherein the target site is selected from the group consisting of a polynucleotide encoding selectable marker resistance, disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein composition, altered oil composition, increased biomass, increased shoot length, increased root length, improved root architecture, modulation of a metabolite, modulation of the proteome, increased seed weight, altered seed carbohydrate composition, altered seed oil composition, altered fatty acid profile, altered seed protein composition, altered seed nutrient composition, improved fertility, improved fecundity, improved environmental tolerance, improved vigor, improved disease resistance, improved disease tolerance, improved tolerance to a heterologous molecule, improved fitness, improved physical characteristic, greater mass, increased production of a biochemical molecule, decreased production of a biochemical molecule, upregulation of a gene, downregulation of a gene, upregulation of a biochemical pathway, downregulation of a biochemical pathway, stimulation of cell reproduction, and suppression of cell reproduction.

9. The method of claim 8, wherein the polynucleotide encodes an altered fatty acid profile.

10. The method of claim 1, wherein the plant is a monocot or a dicot.

11. The method of claim 10, wherein the monocot is selected from the group consisting of maize, rice, sorghum, rye, barley, wheat, millet, oats, sugarcane, turfgrass, and switchgrass.

12. The method of claim 10, wherein the dicot is selected from the group consisting of soybean, canola, alfalfa, sunflower, cotton, tobacco, peanut, potato, tobacco, Arabidopsis, and safflower.

13. The method of claim 1, wherein the editing T-DNA provided to the plant cell is provided via Agrobacterium-mediated transformation.

14. The method of claim 1, wherein the editing T-DNA provided to the plant cell is provided via Ochrobactrum-mediated transformation.

15. The method of claim 1, wherein the editing T-DNA provided to the plant cell is provided via Rhizobiaceae-mediated transformation.

16. The method of claim 1, wherein the guide RNA is operably linked to a plant U6 polymerase III promoter.

17. The method of claim 1, wherein the Cas endonuclease is a plant optimized Cas9 endonuclease.

18. The method of claim 1, wherein the Cas endonuclease gene is operably linked to a nuclear targeting signal upstream of the Cas coding region and a nuclear localization signal downstream of the Cas coding region.

19. The method of claim 1, wherein the target site is located in the gene sequence of a FAD2 gene, a FAD3 gene, or a combination thereof.

20. A plant, plant cell, or seed produced by the method of claim 1.

21. A plant comprising an edited trait of interest, wherein the plant originates from a plant cell comprising an edited trait of interest produced by the method of claim 1.

22. The method of claim 1, wherein the editing T-DNA further comprises a selectable marker expression cassette, a color marker expression cassette, or a combination thereof.

23. The method of claim 1, wherein the Cas endonuclease is expressed by SEQ ID NO:1.

24. A recombinant DNA construct for increasing editing comprising an editing T-DNA for a trait or polynucleotide of interest, wherein the editing T-DNA comprises in operable linkage from a right border to a left border orientation, a guide RNA expression cassette, wherein the guide RNA expression cassette comprises a regulatory element operably linked to a polynucleotide encoding a guide RNA, wherein the guide RNA is complementary to the trait or polynucleotide of interest and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease cleavage in the trait or polynucleotide of interest.

25. The recombinant DNA construct of claim 24, further comprising a trait or polynucleotide of interest modification template cassette, wherein the trait or polynucleotide of interest modification template cassette comprises at least one nucleotide modification of the trait or polynucleotide of interest and the trait or polynucleotide of interest modification template cassette is capable of making at the least one nucleotide modification in the trait or polynucleotide of interest.

26. The recombinant DNA construct of claim 24, further comprises a selectable marker expression cassette, a color marker expression cassette, or a combination thereof.

27. A plant comprising a modified nucleotide sequence, wherein the modified nucleotide sequence was produced by providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary the nucleotide sequence and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage in the nucleotide sequence.

28. The plant of claim 27, wherein the editing T-DNA further comprises a nucleotide modification template cassette, wherein the nucleotide modification template cassette comprises at least one nucleotide modification of the nucleotide sequence and the nucleotide modification template cassette is capable of making at the least one nucleotide modification in the nucleotide sequence.

29. A plant comprising a modified nucleotide sequence, wherein the modified nucleotide sequence was produced by providing to a plant cell an editing T-DNA, wherein the editing T-DNA comprises in operable linkage from a right border to a left border orientation, a guide RNA expression cassette wherein, the guide RNA expression cassette comprises a regulatory element operably linked to a polynucleotide encoding a guide RNA, wherein the guide RNA is complementary a FAD2 genomic sequence in the plant genome, a FAD3 genomic sequence in the plant genome, or a combination thereof and a Cas endonuclease expression cassette, wherein the Cas endonuclease expression cassette comprises a regulatory element operably linked to a Cas endonuclease, wherein the guide RNA and the Cas endonuclease are capable of forming a complex that enables the Cas endonuclease to introduce cleavage in the FAD2 genomic sequence in the plant genome, the FAD3 genomic sequence in the plant genome, or a combination thereof.

30. The plant of claim 29, wherein the editing T-DNA further comprises a polynucleotide modification template cassette, wherein the polynucleotide modification template cassette comprises at least one nucleotide modification of a FAD2 genomic sequence in the plant genome, a FAD3 genomic sequence in the plant genome, or a combination thereof, and the polynucleotide modification template cassette enables the at least one nucleotide modification of the FAD2 genomic sequence in the plant genome, the FAD3 genomic sequence in the plant genome, or the combination thereof.

31. The plant of claim 27 or 29, wherein the plant is a monocot or a dicot.

32. The plant of claim 31, wherein the monocot is selected from the group consisting of maize, rice, sorghum, rye, barley, wheat, millet, oats, sugarcane, turfgrass, and switchgrass.

33. The plant of claim 31, wherein the dicot is selected from the group consisting of soybean, canola, alfalfa, sunflower, cotton, tobacco, peanut, potato, tobacco, Arabidopsis, and safflower.

34. The method of claim 3, the method further comprising a modification template cassette, wherein the modification template cassette comprises at least one nucleotide modification of a FAD2 genomic sequence in the plant genome, a FAD3 genomic sequence in the plant genome, or a combination thereof and the modification template cassette enables the at least one nucleotide modification of the FAD2 genomic sequence in the plant genome, the FAD3 genomic sequence in the plant genome, or the combination thereof.

35. The method of claim 4, wherein the target site is selected from the group consisting of a promoter sequence, a terminator sequence, a regulatory element sequence, a coding sequence, a splice site, a polyubiquitination site, an intron site, an intron enhancing motif, a gene of interest, and a trait of interest.

36. The method of claim 4, wherein the target site is selected from the group consisting of a polynucleotide encoding selectable marker resistance, disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein composition, altered oil composition, increased biomass, increased shoot length, increased root length, improved root architecture, modulation of a metabolite, modulation of the proteome, increased seed weight, altered seed carbohydrate composition, altered seed oil composition, altered fatty acid profile, altered seed protein composition, altered seed nutrient composition, improved fertility, improved fecundity, improved environmental tolerance, improved vigor, improved disease resistance, improved disease tolerance, improved tolerance to a heterologous molecule, improved fitness, improved physical characteristic, greater mass, increased production of a biochemical molecule, decreased production of a biochemical molecule, upregulation of a gene, downregulation of a gene, upregulation of a biochemical pathway, downregulation of a biochemical pathway, stimulation of cell reproduction, and suppression of cell reproduction.

37. The method of claim 36, wherein the polynucleotide encodes an altered fatty acid profile.

38. The method of claim 2, wherein the plant is a monocot or a dicot.

39. The method of claim 3, wherein the plant is a monocot or a dicot.

40. The method of claim 4, wherein the plant is a monocot or a dicot.

41. The method of claim 38, wherein the monocot is selected from the group consisting of maize, rice, sorghum, rye, barley, wheat, millet, oats, sugarcane, turfgrass, and switchgrass.

42. The method of claim 38, wherein the dicot is selected from the group consisting of soybean, canola, alfalfa, sunflower, cotton, tobacco, peanut, potato, tobacco, Arabidopsis, and safflower.

43. The method of claim 39, wherein the monocot is selected from the group consisting of maize, rice, sorghum, rye, barley, wheat, millet, oats, sugarcane, turfgrass, and switchgrass.

44. The method of claim 39, wherein the dicot is selected from the group consisting of soybean, canola, alfalfa, sunflower, cotton, tobacco, peanut, potato, tobacco, Arabidopsis, and safflower.

45. The method of claim 40, wherein the monocot is selected from the group consisting of maize, rice, sorghum, rye, barley, wheat, millet, oats, sugarcane, turfgrass, and switchgrass.

46. The method of claim 40, wherein the dicot is selected from the group consisting of soybean, canola, alfalfa, sunflower, cotton, tobacco, peanut, potato, tobacco, Arabidopsis, and safflower.

47. The method of claim 2, wherein the editing T-DNA provided to the plant cell is provided via Agrobacterium-mediated transformation.

48. The method of claim 3, wherein the editing T-DNA provided to the plant cell is provided via Agrobacterium-mediated transformation.

49. The method of claim 4, wherein the editing T-DNA provided to the plant cell is provided via Agrobacterium-mediated transformation.

50. The method of claim 2, wherein the editing T-DNA provided to the plant cell is provided via Ochrobactrum-mediated transformation.

51. The method of claim 3, wherein the editing T-DNA provided to the plant cell is provided via Ochrobactrum-mediated transformation.

52. The method of claim 4, wherein the editing T-DNA provided to the plant cell is provided via Ochrobactrum-mediated transformation.

53. The method of claim 2, wherein the editing T-DNA provided to the plant cell is provided via Ochrobactrum-mediated transformation.

54. The method of claim 3, wherein the editing T-DNA provided to the plant cell is provided via Rhizobiaceae-mediated transformation.

55. The method of claim 4, wherein the editing T-DNA provided to the plant cell is provided via Rhizobiaceae-mediated transformation.

56. The method of claim 2, wherein the editing T-DNA provided to the plant cell is provided via Rhizobiaceae-mediated transformation.

57. The method of claim 3, wherein the editing T-DNA provided to the plant cell is provided via Rhizobiaceae-mediated transformation.

58. The method of claim 4, wherein the guide RNA is operably linked to a plant U6 polymerase III promoter.

59. The method of claim 2, wherein the guide RNA is operably linked to a plant U6 polymerase III promoter.

60. The method of claim 3, wherein the guide RNA is operably linked to a plant U6 polymerase III promoter.

61. The method of claim 4, wherein the guide RNA is operably linked to a plant U6 polymerase III promoter.

62. The method of claim 2, wherein the Cas endonuclease is a plant optimized Cas9 endonuclease.

63. The method of claim 3, wherein the Cas endonuclease is a plant optimized Cas9 endonuclease.

64. The method of claim 4, wherein the Cas endonuclease is a plant optimized Cas9 endonuclease.

65. The method of claim 2, wherein the Cas endonuclease gene is operably linked to a nuclear targeting signal upstream of the Cas coding region and a nuclear localization signal downstream of the Cas coding region.

66. The method of claim 3, wherein the Cas endonuclease gene is operably linked to a nuclear targeting signal upstream of the Cas coding region and a nuclear localization signal downstream of the Cas coding region.

67. The method of claim 4, wherein the Cas endonuclease gene is operably linked to a nuclear targeting signal upstream of the Cas coding region and a nuclear localization signal downstream of the Cas coding region.

68. The method of claim 17, wherein the Cas endonuclease gene is operably linked to a nuclear targeting signal upstream of the Cas coding region and a nuclear localization signal downstream of the Cas coding region.

69. The method of claim 62, wherein the Cas endonuclease gene is operably linked to a nuclear targeting signal upstream of the Cas coding region and a nuclear localization signal downstream of the Cas coding region.

70. The method of claim 63, wherein the Cas endonuclease gene is operably linked to a nuclear targeting signal upstream of the Cas coding region and a nuclear localization signal downstream of the Cas coding region.

71. The method of claim 64, wherein the Cas endonuclease gene is operably linked to a nuclear targeting signal upstream of the Cas coding region and a nuclear localization signal downstream of the Cas coding region.

72. A plant, plant cell, or seed produced by the method of claim 2.

73. A plant, plant cell, or seed produced by the method of claim 3.

74. A plant, plant cell, or seed produced by the method of claim 4.

75. A plant, plant cell, or seed produced by the method of claim 5.

76. A plant comprising an edited trait of interest, wherein the plant originates from a plant cell comprising an edited trait of interest produced by the method of claim 2.

77. A plant comprising an edited trait of interest, wherein the plant originates from a plant cell comprising an edited trait of interest produced by the method of claim 3.

78. A plant comprising an edited trait of interest, wherein the plant originates from a plant cell comprising an edited trait of interest produced by the method of claim 4.

79. The method of claim 2, wherein the editing T-DNA further comprises a selectable marker expression cassette, a color marker expression cassette, or a combination thereof.

80. The method of claim 3, wherein the editing T-DNA further comprises a selectable marker expression cassette, a color marker expression cassette, or a combination thereof.

81. The method of claim 4, wherein the editing T-DNA further comprises a selectable marker expression cassette, a color marker expression cassette, or a combination thereof.

82. The method of claim 2, wherein the Cas endonuclease is expressed by SEQ ID NO:1.

83. The method of claim 3, wherein the Cas endonuclease is expressed by SEQ ID NO:1.

84. The method of claim 4, wherein the Cas endonuclease is expressed by SEQ ID NO:1.

85. The plant of claim 29, wherein the plant is a monocot or a dicot.

86. The plant of claim 85, wherein the monocot is selected from the group consisting of maize, rice, sorghum, rye, barley, wheat, millet, oats, sugarcane, turfgrass, and switchgrass.

87. The plant of claim 85, wherein the dicot is selected from the group consisting of soybean, canola, alfalfa, sunflower, cotton, tobacco, peanut, potato, tobacco, Arabidopsis, and safflower.
